SEQUENCE LISTING (24 sequences)

SEQ ID NO: 1 - DNA, 5620 bp, Zea mays
cactagagtaaactagttagtccaaatatttgtgttgggcattcaaccaccaaaatttaattagggactaggtgtaatcctaattccctttcataaactctatcccgtcgagtccgtttgcttgtgacttagtttgggaccctccttttctattgaaaggggttcagatcgctggggaccaagactgaaggaaaaggcgtttgtcctctagggtggtccggagctgtgtagccgcttagcctggtcctgtaccctaagcctacatacttcaccactcttgaacaggtggcctattacctggaactgggtccctagaggcccggacctccttctagcttacaggttggcttcctaaactctgctcgtcaagcccggttgcgcatctcaaaggatcaagtgcaacagagtgtgggtcccatgggtgtgttactaaacatcaatcttgagtcatatgcagggcaaaatcctggttaggcggtagccgaggcgtcgggtagaacaccaagagacacccacaccaagtgctagcgacctaaaccatgacatgggggctcgggcccaccagtcatatgtagtgggtggcaggcgtaggtgtgaaaaggcgggatatgaaccgtcgggcgcacgacacacacaatgagtaccttctcaggaatccctgtagtgataaaaaccgattttactgccacgatggtggccaacgttatgcccatgaaccaggcgtgggagccgcaggtcaatgagctgcatcgacataagcgacagtaagtgatctgacggtaagcgtaacagcgagcgtgacaccgttggcgctagtcacaatcaagttgcttgtagcaggatcacagttatgccccccccacttgcgagttcgtaacctctcccgaggtgggctcgggggccactgtcgataccccgtaactagggtactctctcctactgtgtcaaggcagatacctgtgcggttagctctaaccacatgctaaagaactaagcagccgggcccacgggaccggacctcgactgacaaggaccgctggccttggaactcgctccctgctcagggacagatccggtgctgccacgtgtcctagaagagaagatgcctagggactactcccataggtctggacccatgggtggggcccgaatccccacgtatgtcgtccggaccccatgggcgggtcctgaatctccacgtatgtcgttcgaatttattgtattgcattgtgtgtagtgtcgtattattggacattcttttctttcggttgaactgcgtatatattgttaggattgaattttgtgtattgatggatttatcattgaaatttgtgattttttattgctgattgtagcaatgagagcacaaagcagtgtaaaactcgactcactgaagaattctatgagatattaaggtcaatgattatatatattatttatttttatacattaattatgcactaacatttttattaaataatttatgtgtgattgtaacagcatggacatcgtacctgttacaatataatagccctccctttacttcattcatgttctgcattccggtctggtcgtctggagaggaggtctcctatgttctgcgagacagcgagagcgatacagtagtctggtacgcctcttcccgtatccgtagcgtcgacgctgctgcctgctgggaaaaattcgtgggctgaccgttgccacaaacggcccaagcgacgcaagcctccctgcaggctgcagcacggagctcgctcgctgctatctgtcttagccatccgtccgcagcctatccaattccattccaaatccgcagtccgcagacgagagtggaagcggggcacagacaggaagagaagccaatggcgctgtccctctccctcgcgcgccccgcgcccctcgccgtttccgccggcgcaggagccaggaagctacccgcagccagcctcgcattcccggcgaaatccttcttcggcgcgccgctggccgccaccgcggcctccgtcgcgtcgccgctcccgcgcaagccggccacctccaccacctcgctcgaggtcgtcgcggcggggaagaagggctacaagatgaagacgcacaaggttctttcctgctatctcgtccttggttcctgagagtcatcgggttgttcggtgccttttgctgattcgtgtctcggtcggttctttgtgtgtcgtcgctcgtcaaggcgtcggcgaagcggttccgggtgacggggaggggcaagatcgtgcggcggtgcgccgggaagcagcacttgctcgccaagaagaacaccaagcgcaagaagaggctctcgaagatggtattcttctttcctccctccactcccactctagtgccgctcaaattttgttcagctgcttcttatgtcgcagctaatgacggcttgcatattagttattgcctgttttaatagctgtacatagaaaacaagtccggtgcgtctccgtaaattagatgctaatgtcgatttttttatgtcaatagcacattgcgctctcgtattttaattgctcgataggtttattgtgataacgttgctggaaactggaaaggcgaaatggagggacgtccggactggggtttgttttgctgatgtgctctggggtgctacctttgttactttcttggatctcgttgtctcctactcattaggatgcacagtctgttcctcgatcatcaaatgtgtgtcagggccaatagatggcatgaaaaagcttttggctacaattgaacttgtcgaagttcacagattattatcatggtccttatataacgtttgtttctgaaaatgaactgttttacttgtacacataaaattacctctggaaataggcattacaattttcagaaagaagtgggtgtgggtgtaaaacgtactgacctatgaacatttctagttttactccactattgttagtcagctaatgtattgggggagtgctgcccatgaattagacatcctgttcttccatttttgtgttgtgataataactttcagattcatcaagaatgtaagggtcttagaagttagaacttgttctactgtagaatttggtgataattgaatagcgaggatatttctcctcgatggatcccctccatttacctgtccaccaaaccagcactaggaatcctgatttcccactaatctgcactttagtttttttctgattaagtccagtctttcccatccttcacgcatccttcacgatgaatgctgtagaatttggttatgtcattcttatgaatcctgatttcccactaatctgcacttcagttagtgtgtgcttagctgctttcttttgtgaaccgggctatattagagaggtgtagttttttcagattaagacattaagtccagtctttaccatccttcacgcattcatcgtagtagtctgatgtactgcttcacacacacacacctagcagttgatgttggcatcccatgggcatggggtgaagcaaaatttctatttttttgtgacagaaaaattattaacctctgtccataaagttgcgggccattttcaatgtatccttgaaaccgtggcaattccatttgcagtctgaacttgacgttttcttttcttcttaaatcacactgctatatatttttttcaggtgcaagtcaacaagagtgactacgacaatgttacgggtgcactgccctacctcaaagtgaataggaaagcaaactgagagctacgtggtcttcaaaaaatcattaagtttcgttccaccaaattgtaattttgtgtatcttccactgtatttccttctcaaaaatactgaggcatcatttcaaagcaagcaaaaaacaactcctggtatcaacagtatagcgatatttcagaatgaggtgcactgcttgctatagttgttatttagtcgcaaatatgtgcaagtaagagtggcacttgagccattagctcctcttaggatatggccagcagatcgtgtatatggctgtgcaagacactgttttgcaccattttatatatagactgcattgtttacatgatgaagcttgaaataggatatgggatgagtgcaactgctggagatagcctcagggagctaaacttttcactggtctgtcttaaaccttaatctaatggttttcatctgtattgctggctgccaaattgtggacgttttttgggtaaccagtttcatgaccaaatttcagtgacgccaatgcttgtctttaaaaaaatcgtattttgcaaaaaaaaatttgtagtcaccaaccaaatatgtgtaacaaactttctttcctctgctttgtgatctccaagttcatcttaactatctgctttgggaataacaaatgatcgattcggccctatttagctctccttttcctaaaacccacaaacctatttgtagttttgtacaccgccatagctgtcaacaatgaatgttgatccgagatagggagagggtgaaatcacgagataaacctaaacataccatctgactgttatctagtattctactaaccaacctaaagagttttgctggctagaacaaatcctatgatctaacctatgcactgctttaggaagtaaaggcacataaaacacacaagtgctagctcgcggatctctattccattttgaagctccacagaggatataattttggtcacaactcataaggtttgtcagatcacggtcttgagctcgctgcaatgcgtcatgaaagggtctcctatcgctgaataagtgtgttcatgatgttccatacaacgcttgagggtagatatatcgatagtgcattagtgtaacgtgttgaccctttttagtactatgacaaagggtcgtggtcagcaatggaccatcatgtttgttttggttaataggtggattcatctatatttataccactatataatctattcgtctaaattttacaaaacccacccaaccaaatcatctccacacccatgacctccattaaatccgtcaaacaaagtgcacccagacttaacacgccatcaaaactaacagttcagattgctgaataaccccttctcccttactcacccgaatccaatggacaagattatttcgatcatccttcatccctacccctcccctgcgactgaaagggaaatagggttaacattttcccagtattaattttggtggttgaatgcccaacacaaatattggactaactagtttgctctagattatatattctacaggtgcataaaggttcaacacaaaccaataaaagatcaaagttagggttcaaaaacaaaagagcaaagaaaccgagtgtgccctggtctggcgcaccggactgtccggtgcaccaggaccgtacagttccaaactcgccacccttgggtttctctggacgtgctccgctataattcactggaatgtccggtgcaccagcggagcaacggctacttcgcgcaatggtcgcctgcaaaaaagctgaagaatcagatgaacagtgcgcggcagtgcgcggcagagcagagccgcccgtcagaggcgcaccggacactgcgcagtgcatgtctggtgccactagaagacaaagcctccaatggtcagaggctcccgaaccctaacggttg

SEQ ID NO: 2 - DNA, 2004 bp, Zea mays
ggcggggaagaagggctacaagatgaagacgcacaaggttctttccctgctatctcgtccttggttcctgagagtcatcgggttgttcggtgccttttgctgattcgtgtctcggtcggttctttgtgtgtcgtcgctcgtcaaggcgtcggcgaagcggttccgggtgacgcggaggggcaagatcgtgcggcggtgcgccgggaagcagcacttgctcgccaagaagaacaccaagcgcaagaagaggctctcgaagatggtattcttctttcctccctccactcccactctagtgccgctcaaattttgttcagctgcttcttatgtcgcagctaatgacggcttgcatattagttattgcctgttttaatagctgtacatagaaaacaagtccggtgcgtctccgtaaattagatgctaatgtggatttttttatgtcaatagcacattgcgctctcgtattttaattgctcgataggtttattgtgataacgttgctggaaactggaaaggcgaaatggagggacgtccggactggggtttgttttgctgatgtgctctggggtgctacctttgttactttcttggcatctcgttgtctcctactcattaggatgcacagtctgttcctcgatcatcaaatgtgtgtgagggccaatagatggcatgaaaaagcttttggctacaattgaacttgtcgaagttcacagattattatcatggtccttatataacgtttgtttctgaaaatgaactgttttacttgtacacataaaattacctctggaaataggcattacaattttcagaaagaagtgggtgtgggtgtaaaacgtactgacctatgaacatttctagttttactccactattgttagtcagctaatgtattgggggagtgctgcccatgaattagacatcctgttcttccatttttgtgttgtgataataactttcagattcatcaagaatgtaagggtcttagaagttagaacttgttctactgtagaatttggtgataattgaatagcgaggatatttctcctcgatggatcccctccatttacctgtccaccaaaccagcactaggaatcctgatttcccactaatctgcactttagtttttttctgattaagtccagtctttcccatccttcacgcatccttcacgatgaatgctgtagaatttggttatgtcattcttatgaatcctgatttcccactaatctgcacttcagttagtgtgtgcttagctgctttcttttgtgaaccgggctatattagagaggtgtagttttttcagattaagacattaagtccagtctttaccatccttcacgcattcatcgtagtagtctgatgtactgcttcacacacacacacacctagcagttgatgttggcatcccatgggcatggggtgaagcaaaatttctatttttttgtgacagaaaaattattaacctctgtccataaagttgcgggccattttcaatgtatccttgaaaccgtggcaattccatttgcagtctgaacttgacgttttcttttcttcttaaatcacactgctatatatttttttcaggtgcaagtcaacaagagtgactacgacaatgttacgggtgcactgccctacctcaaagtgaataggaaagcaaactgagagctacgtggtcttcaaaaaatcattaagtttcgttccaccaaattgtaattttgtgtatcttccactgtatttccttctcaaaaatactgaggcatcatttcaaagcaagcaaaaaacaactcctggtatcaacagtatagcgatatttcagaatgaggtgcactgcttgctatagttgttatttagtcgcaaatatgtgcaagtaagagtggcacttgagccattagctcctcttaggatatggccagcagatcgtgtatatggctgtgcaagacactgttttgcaccattttatatatagactgcattgtttacatgatgaagcttgaaatagga

SEQ ID NO: 3 - DNA, 833 bp, Zea mays
gcagtccgcagacgagagtggaagcggggcacagacaggaagagaagccaatggcgctgtccctctccctcgcgcgccccgcgcccctcgccgtttccgccggcgcaggagccaggaagctacccgcagccagcctcgcattcccggcgaaatccttcttcggcgcgccgctggccgccaccgcggcctccgtcgcgtcgccgctcccgcgcaagccggccacctccaccacctcgctcgaggtcgtcgcggcggggaagaagggctacaagatgaagacgcacaaggcgtcggcgaagcggttccgggtgacggggaggggcaagatcgtgcggcggtgcgccgggaagcagcacttgctcgccaagaagaacaccaagcgcaagaagaggctctcgaagatggtgcaagtcaacaagagtgactacgacaatgttacgggtgcactgccctacctcaaagtgaataggaaagcaaactgagagctacgtggtcttcaaaaaatcattaagtttcgttccaccaaattgtaattttgtgtatcttccactgtatttccttctcaaaaatactgaggcatcatttcaaagcaagcaaaaaacaactcctggtatcaacagtatagcgatatttcagaatgaggtgcactgcttgctatagttgttatttagtcgcaaatatgtgcaagtaagagtggcacttgagccattagctcctcttaggatatggccagcagatcgtgtatatggctgtgcaagacactgttttgcaccattttatatatagactgcattgtttacatgatgaagcttgaaataggatatgggatgagt

SEQ ID NO: 4 - DNA, 621 bp, Zea mays
ctacccgcagccagcctcgcattcccggcgaaatccttcttcggcgcgccgctggctgccaccgcggcctccgtcgcgtcgccgctcccgcgcaagccggccacctccaccacctcgcttgaggtcgtcgcggcggggaagaagggctacaagatgaagacgcacaaggcgtcggcgaagcggttccgggtgacggggaggggcaagatcgtgcggcggtgcgccgggaagcagcacttgctcgccaagaagaacaccaagcgcaagaagaggctctcgaagatggtgcaagtcaacaagagtgactacgacaatgttacgggtgcactgccctacctcaaagtgaataggaaagcaaactgagagctacgtggtcttcaaaaaatcattaagtttcgttccaccaaattgtaattttgtgtatcttccactgtatttccttctcaaaaatactgaggcatcatttcaaagcaagcaaaaaacaactcctggtatcaacagtatagcgatatttcagaatgaggtgcactgcttgctatagttgttatttagtcgcaaatatgtgcaagtaagagtggcacttgagccattagctcctcttaggatatggccagcagatcg

SEQ ID NO: 5 - protein, 143 aa, Zea mays
MALSLSLARPAPLAVSAGAGARKLPAASLAFPAKSFFGAPLAATAASVASPLPRKPATSTTSLEVVAAGKKGYKMKTHKASAKRFRVTGRGKIVRRCAGKQHLLAKKNTKRKKRLSKMVQVNKSDYDNVTGALPYLKVNRKAN

SEQ ID NO: 6 - protein, 143 aa, Zea mays
MALSLSLARPAPLAVSAGAGARKLPAASLAFPAKSFFGAPLAATAASVASPLPRKPATSTTSLEVVAAGKKGYKMKTHKASAKRFRVTRRGKIVRRCAGKQHLLAKKNTKRKKRLSKMVQVNKSDYDNVTGALPYLKVNRKAN

SEQ ID NO: 7 - DNA, 166 bp, Zea mays
ggcggggaagaagggctacaagatgaagacgcacaaggttctttcctgctatctcgtccttggttcctgagagtcatcgggttgttcggtgccttttgctgattcgtgtctcggtcggttctttgtgtgtcgtcgctcgtcgagataattgccattatggacgaag

SEQ ID NO: 8 - DNA, 20 bp, Artificial Sequence
  Primer
tcyataatggcaattatctc

SEQ ID NO: 9 - DNA, 17 bp, Artificial Sequence
  Primer
gatgagtcctgagtaan

SEQ ID NO: 10 - DNA, 28 bp, Artificial Sequence
  Primer
gaaactggaaaggcgaaatggagggacg

SEQ ID NO: 11 - DNA, 32 bp, Artificial Sequence
  Primer
gctcgataggtttattgtgataacgttgctgg

SEQ ID NO: 12 - DNA, 28 bp, Artificial Sequence
  Primer
gtccctccatttcgcctttccagtttcc

SEQ ID NO: 13 - DNA, 32 bp, Artificial Sequence
  Primer
ccagcaacgttatcacaataaacctatcgagc

SEQ ID NO: 14 - DNA, 31 bp, Artificial Sequence
  Primer
gagaagccaacgccawcgcctcyatttcgtc

SEQ ID NO: 15 - DNA, 27 bp, Artificial Sequence
  Primer
ggatcctaatacgactcactatagggc

SEQ ID NO: 16 - DNA, 18 bp, Artificial Sequence
  Primer
ctatagggctcgagcggc

SEQ ID NO: 17 - DNA, 25 bp, Artificial Sequence
  Primer
ctacccgcagccagcctcgcattcc

SEQ ID NO: 18 - DNA, 30 bp, Artificial Sequence
  Primer
ggcggggaagaagggctacaagatgaagac

SEQ ID NO: 19 - DNA, 29 bp, Artificial Sequence
  Primer
gccaagaagaacaccaagcgcaagaagag

SEQ ID NO: 20 - DNA, 25 bp, Artificial Sequence
  Primer
cgatctgctggccatatcctaagag

SEQ ID NO: 21 - DNA, 24 bp, Artificial Sequence
  Primer
catcttcgagagcctcttcttgcg

SEQ ID NO: 22 - DNA, 26 bp, Artificial Sequence
  Primer
cttcatcttgtagcccttcttccccg

SEQ ID NO: 23 - DNA, 23 bp, Artificial Sequence
  Primer
gcacccgtaacattgtcgtagtc

SEQ ID NO: 24 - DNA, 25 bp, Artificial Sequence
  Primer
cttcgtccataatggcaattatctc

(19) United States
(12) Patent Application Publication
(10) Pub. No.: US 2005/0086716 A1
(43) Pub. Date: Apr. 21, 2005

(54) Nucleic acids coding for a ISUM2A polypeptide and use of said nucleic acids to obtain transformed plants producing seeds altered in germ development

(76) Inventors: Peter Rogowsky, Lyon (FR); Thierry Heckel, Lutzelbourg (FR); Pascual Perez, Chanonat (FR)

Correspondence Address: BAKER & BOTTS, 30 ROCKEFELLER PLAZA, NEW YORK, NY, 10112, US

(21) Appl. No.: 10/759,548

(22) Filed: Jan. 16, 2004

Related U.S. Application Data
(63) Continuation of application No. PCT/FR02/02553, filed on Jul. 17, 2002.

(30) Foreign Application Priority Data
Jul. 18, 2001 (FR) FR 01/09,631

(51) Int. Cl. C12N 15/82; G01N 33/53; A01H 5/00; C12Q 1/68

(52) U.S. Cl. 800/281; 536/23.6; 435/6; 435/320.1; 435/419; 800/298; 800/278; 530/370; 530/387.1; 435/7.1

(57) ABSTRACT

The invention provides nucleic acid sequences and polypeptides whereof the expression is essential for the development of the embryo in a plant seed, and whereof the deficient expression results in the production of seeds altered in germ development.

This application is a continuation of International Patent application No. PCT/FRO2/02553 filed Jul. 17, 2002 and published in French as WO 03/008585 on Jan. 30, 2003, which claims priority to French Patent Application No. FR 01/09631 filed Jul. 18, 2001.

### FIELD OF THE INVENTION

The present invention relates to the field of improving the agronomic characteristics of plants for the purpose of obtaining plant transformation products with improved characteristics for industry, most particularly for the agrofoods industries, for example for the production of starch, plant oil or semolina.

### BACKGROUND OF THE INVENTION

In general, there exists a need, in the state of the art, to improve the agronomic, dietary or industrial qualities of grains, in particular for industries producing starch, plant oil or semolina. The grain embryo is the compartment of the grain from which plant oil is extracted. It would be industrially advantageous to obtain plants in which the grains have an overdeveloped embryo rich in oil. Conversely underdeveloped embyros would be particularly advantageous for the production of semolina. It would also be advantageous for the starch industry to obtain plants in which the grains lack germs, the grains consisting essentially of the starch-rich albumen.

In general, there exists a need, in the state of the art, for isolation and characterization of regulatory sequences allowing specific expression of a nucleic acid of interest in the embryo and/or the albumen, early in the course of the plant's development, in order to modulate the agronomic qualities of the mature grain.

The amount of starch produced today, for multiple industrial applications, is approximately 1 billion tonnes annually throughout the world. Starch represents a raw material which is used in industries as diverse as the food industry, pharmacy and the paper industries, but also in the microbiological field, where it can be used as a nutritive substrate.

Conventionally, starch is obtained from the grains of large crop cereal plants such as wheat, maize and sorghum. The grains consist essentially of a germ associated with the albumen. The starch is entirely contained in the albumen, while the germ is rich in oil. As a result of this, the processes for preparing starch used in the starch industry necessarily comprise a step for separating the germ, which is rich in oil, from the albumen.

It would be particularly advantageous to eliminate the step separating the germ from the albumen in starch purification processes. This would make it possible to significantly simplify these processes and also to make them more rapid and less expensive.

There also exists the need in the art to improve the agronomic qualities of the grains used in processes for converting the vitreous portion of the maize grain (kernel) into semolina. A milling process makes it possible to finely separate the various constituents of the grain in order to satisfy the users' requirements. It comprises in particular a drying step which should be as gentle as possible in order to avoid migration of the lipids from the germs to the kernel (migration to the kernal is prejudicial to the subsequent quality of the semolinas), and a degerming step, carried out by fragmentation. The aim of the degerming step is to carefully separate the germs in order to avoid the lipids that they contain ending up in the semolinas.

Grains altered in germ development according to the invention, therefore, offer a definite advantage to the semolina industry since they facilitate the industrial process (gain in time and yield) by eliminating this ‘contamination’ of the kernel with the lipids from the germ. In addition, grains of the invention provide good quality and a good lifespan of the products, this being a function of the residual of fats (<0.8% is a standard in the profession for ‘noble’ products: hominy, grits, semolinas and flours for human food).

The semolinas produced may be used almost exclusively in human foodstuffs (beers, breakfast cereals, crackers, polenta, tortilla, corn chips, food flours, etc).

By way of examples, the grains according to the invention may be used for the production of breakfast cereals or ‘cornflakes’, which constitute a market which has, for 15 years, been experiencing an annual average increase of the order of 20%. Two processes are conventionally used: a conventional rolling process using hominy (the largest semolinas completely degermed and calibrated) or a cooking-extrusion process using semolinas with a specific particle size.

According to another embodiment of the invention, it is possible to obtain grains with large embryos, which is desired in the oil industry. Corn oil is commonly used for human food, but may also be used by the pharmaceutical industry and the cosmetics industry. The cakes are, themselves, exploited in animal feedstuffs either directly, or else remixed with corn-gluten feed.

Many plants carrying mutations affecting both the embryo and the albumen of the grain are known in the state of the art. These mutant plants are conventionally referred to as “dek” (for “defective kernel”) mutants.

On the other hand, there are very few maize mutants which are altered by a deficiency in the development of the embryo of the grain but which leaves the albumen intact.

These are essentially the mutant plants observed by Sheridan et al. (1993, 1995) and those described by Elster et al. (2000). However, these authors provide no result regarding cosegregation of genetic markers with the mutant phenotype.

Heckel et al. (1999) have analyzed five maize mutants affected at various stages of embryo development. A first group of mutants comprises mutants which produce pro-embryonic structures resembling those produced in the wild-type plants, but the pro-embryos do not reach the subsequent developmental stages.

In the second group of mutants, the mutants emb*-8522 and emb*-8535 are altered by a complete deficiency in apical-basal differentiation, whereas, in the mutant embb*-8516, the authors have observed embryo-like structures arising from the suspensor.

An analysis of cosegregation of the emb *-8516 and emb *-8522 mutations enabled Heckel et al. to determine that the mutant phenotype cosegregated with the presence of a transposon. However, the molecular characterization of the various mutations was not described. In particular, the mutation affecting the emb*-8516 mutant plant could not be located on any of the maize chromosomes.

### SUMMARY OF THE INVENTION

The invention provides nucleic acid sequences, the expression of which are essential for embryo development in a plant grain, and a deficiency in the expression of which leads to the production of grains altered in germ development. The invention also provides nucleic acids and polypeptides that correspond to these nucleic acid sequences.

A subject of the invention is a nucleic acid comprising a polynucleotide encoding an ISUM2A polypeptide, chosen from sequences having at least 95% amino acid identity with the sequences SEQ ID NO:5 and SEQ ID NO:6, or encoding a fragment of an ISUM2A polypeptide. Another subject of the invention is nucleic acids complementary to a polynucleotide encoding an ISUM2A polypeptide, chosen from the sequences having at least 95% amino acid identity with the sequences SEQ ID NO:5 and SEQ ID NO:6, or encoding a fragment of an ISUM2A polypeptide.

Preferably, the nucleic acid encoding the ISUM2A polypeptide, or encoding a fragment of this polypeptide, also comprises an expression control sequence capable of regulating the synthesis of the ISUM2A polypeptide, the expression control sequence preferably being sensitive to the action of an inducing signal.

The expression control sequence may be equally a transcription- or translation-repressing sequence or, on the contrary, a transcription- or translation-activating sequence.

The invention also relates to methods for obtaining a transformed plant capable of producing grains with altered germ development, and also parts of such a plant, in particular its seeds.

A subject of the invention is also a product of transformation of a seed with altered germ development produced by said transformed plant, preferably a starch.

A subject of the invention is also the ISUM2A polypeptide, or a fragment of this polypeptide, encoded by a nucleic acid as defined above, and also antibodies directed against the ISUM2A polypeptide.

The invention also relates to methods for detecting the presence of the ISUM2A polypeptide in a sample, and to methods for detecting the presence of a nucleic acid encoding this polypeptide in a sample.

### BRIEF DESCRIPTION OF THE DRAWINGS

The present invention is also illustrated, but without limitation, by the following figures:

FIG. 1 illustrates the genomic nucleotide sequence of the isum2A gene present in the maize plant denoted HD5×HD7 (SEQ ID NO:1). The three exons of the isum2A gene are represented in the form of boxes, under the corresponding nucleotide sequence. The various recognition sites for restriction endonucleases are indicated, above the corresponding nucleotide sequence.

FIG. 2 illustrates a nucleotide sequence of the cDNA of the isum2A gene in the maize plant denoted HD5×HD7 (SEQ ID NO:3). The sequences derived from the three exons of the gene are represented by boxes located under the corresponding nucleotide sequence. The deduced amino acid sequence (SEQ ID NO:5) is represented below the boxes representing the exons. The box denoted “RL35 domain” corresponds to the portion of the sequence of the ISUM2A polypeptide that is homologous with certain chloroplast proteins.

FIG. 3 illustrates a partial cDNA sequence (SEQ ID NO:4) and deduced amino acid sequence (SEQ ID NO:6) corresponding to the messenger RNA found in the maize plant denoted Al 88. The boxes located below the peptide sequence represent the three exons derived from the isum2A gene, found in the maize line A188. The box denoted “RL35 domain” corresponds to the portion of the ISUM2A polypeptide having homology with chloroplast proteins.

FIG. 4 illustrates the exon and intron structure of the isum2A gene, the boxes representing the exons of the gene. The base A of the ATG codon is the nucleotide at position 51 of the first exon and the base T of the TGA translation stop codon is the nucleotide at position 76 of the third exon. The boxes located below the structure of the isum2A gene represent the various genomic DNA clones used in the examples. The boxes located above the structure of the isum2A gene represent the various cDNA clones used in the examples.

FIG. 5 illustrates a map of the plasmid pRDP5.

FIG. 6 illustrates the map of the plasmid pTRE sold by the company CLONTECH LABORATORIES Inc., the structure of which is accessible in the GENBANK database under the accession number U89931.

FIG. 7 illustrates the map of the plasmid pDM302 described by CAO et al. (1992).

FIG. 8 illustrates the map of the plasmid pTet-Off sold by the company CLONTECH (reference in catalog for year 2000: K1620-A).

FIG. 9 illustrates the map of the plasmid pRDP4.

FIG. 10 illustrates the map of the plasmid pTA7001 described by AOYAMA et al. (1997).

FIG. 11 illustrates the map of the plasmid pRDP2.

FIG. 12 illustrates the map of the plasmid pRDP3.

FIG. 13 illustrates the principle for the functioning of the expression system II summarized in Table 3 and which is the subject of Example 2.

FIG. 13A illustrates the constitutive expression of the ISUM2A polypeptide under the effect of activation of the promoter containing the TRE sequence by the tTA activator, which is itself expressed constitutively.

FIG. 13B illustrates the repression of the synthesis of the ISUM2A polypeptide when the tTA activator is brought into contact with tetracycline, which deactivates it and prevents it from activating the promoter containing the TRE sequence.

FIG. 14 illustrates a scheme for the functioning of an expression system I summarized in Table 3 and which is the subject of Example 3.

FIG. 14A illustrates the synthesis of the ISUM2A polypeptide when the promoter containing the UAS sequence is activated by the GVG fusion protein, in the presence of a glucocorticoid.

FIG. 14B illustrates the absence of production of ISUM2A polypeptide in a situation in which the GVG fusion protein is produced in the absence of glucocorticoid and does not activate the UAS sequence of the promoter controlling the expression of the isum2A gene.

### BRIEF DESCRIPTION OF THE SEQUENCES

A brief description of the sequences is provided in Table 13.

### DETAILED DESCRIPTION OF THE INVENTION

According to the invention, a population of 25 000 plants having been subject to a high degree of mutagenesis by the random insertion, into their genome, of the mutator transposon described by Bennetzen, J. L. P. S. Springer, A. D. Cresse, and M. Hendrickx (1993), Chandler, V. L. and K. J. Hardeman (1992) has been generated. The plants were placed in culture before analyzing their DNA.

For one of the mutant plants, designated G2422, it was shown that the insertion of the mutator transposon was located in the first intron of a particular gene, this gene having been designated isum2A, at a distance of 3 bp from the second exon of this gene.

On another mutant plant, the inventors observed cosegregation between the presence of a “germ-free grains” phenotype and the insertion of the mutator transposon into this same isum2A gene.

The isum2A gene, which is shown, according to the invention, to be necessary for the normal development of the embryo of the grain, was then isolated and completely characterized.

The isum2A gene comprises three exons and two introns. The open reading frame begins in the first exon and ends in the third exon of this gene.

A partial product of transcription of the isum2A gene was also isolated and characterized, and the structure of a large part of the ISUM2A protein was deduced from the cDNA corresponding to the transcription product of the gene.

It has been shown, according to the invention, that the isum2A gene is expressed in the embryo and the albumen 12 days after pollination, and also in the leaves and the roots.

In addition, the inventors have shown that insertion of the mutator transposon into intron No. 2 of the isum2A gene blocked its expression, since the presence of its transcription product was no longer detectable in an albumen of a plant homozygous for the interruption of the isum2A gene (isum2A::Mu/isum2A::Mu).

It has thus been shown, according to the invention, that plants homozygous for a mutation in the gene encoding the ISUM2A polypeptide produce grains consisting essentially of the albumen, and having altered germ development. The germ is also called embryo.

The inventors have also shown that the seeds representing a quarter of the grains produced by heterozygous plants, and in which both copies of the isum2A gene comprise a mutation, do not allow a viable and fertile plant to be obtained. It has more particularly been shown, according to the invention, that the seeds representing a quarter of the seeds produced by plants heterozygous for the mutated allele(s) resulting in the recessive mutation of the “emb” type would not make it possible to obtain a viable and fertile plant.

In order to remedy this drawback, the invention also provides complementation systems which allow the expression of a nucleic acid encoding a functional ISUM2A polypeptide in plants in which the two alleles of the isum2a gene are mutated. It is possible for these complementation systems to be made inducible so as to precisely control, over time, the expression of a nucleic acid encoding an ISUM2A polypeptide. Thus, the invention provides methods to obtain viable plants which produce grains with altered germ development. These grains may be directly used in industry, particularly in the starch industry and in the semolina industry, without a step for separating the embryo.

According to another aspect, the invention also provides the means for obtaining plants which produce grains altered in germ development, in which the germ is enriched in oil due to early expression or overexpression of the ISUM2A polypeptide.

The invention relates to nucleotide sequences involved in embryo development, and to their use in molecular constructs intended to improve the agronomic, food or industrial quality of a plant, modulating in particular the size of the embryo and/or its development.

In fact, an early and specific action on the development of the tissues of the embryo and of the albumen may be desired:
(1) According to a first embodiment, it may be possible to obtain grains or fruits enriched in oil (large embryo), via the use of promoters which direct the expression of the nucleic acids according to the invention either constitutively or early in grain development, and more particularly in the germ.
(2) According to another embodiment, albumen with altered germ development may also be obtained according to this model, for industrial applications in starch production and semolina production.

The invention is therefore also directed toward methods for modifying the agronomic and/or nutritional qualities of a plant, by a targeted and early action on the development of the embryo, using transformation of the plants with a vector according to the invention. In particular, it is directed toward modifying the size and/or the development of the embryo. It is also directed toward altering the development of the embryo, for the purpose of producing embyro-free grains for cereals in particular, which are of value for the starch and semolina industries.

A subject of the invention is also the use of an expression cassette as defined above, for obtaining a transgenic angiosperm plant exhibiting improved agronomic or nutritional qualities.

Advantageously, the transgenic plant obtained can produce grains with modified oil contents or with altered germ development, in comparison with a nontransformed plant.

The invention also relates to the use of the transgenic plants obtained according to the invention, or parts of these plants, in particular seeds, grains and fruits, for preparing derived products, in particular food products.

Also part of the invention are the products obtained, whether they are seeds, seed meals or grains enriched in oil or seeds with altered germ development, which are suitable for the semolina industry.

The invention also relates to any composition for human or animal food prepared from said obtained products.

According to another aspect, the invention relates to the use of the sequences and allelic variants defined according to the invention, in selection programs aimed at producing plants with an embryo modified in terms of size and/or development having an influence on the starch/oil content. These sequences can in particular be used in experiments for mapping and colocalizing QTLs for the oil content or the size of the embryo, in order to define the most advantageous allelic variants for a selection approach, comprising:
genotyping individuals by means of nucleic acid probes or primers obtained from sequences and allelic variants described according to the invention;
selecting, from these individuals, plants which comprise a high frequency of favorable alleles associated with the size and/or with the development of the embryo.
   Isum2A Gene Nucleic Acids

The nucleotide sequence of the isum2A gene comprises, from the 5′ end to the 3′ end, (i) a noncoding upstream sequence potentially carrying regulatory elements for transcription and/or translation of the gene, (ii) a coding sequence comprising the three exons and the two introns of the gene and (iii) a noncoding sequence located downstream of the final exon of the gene. The sequence of the isum2A gene according to the invention is referenced as the sequence SEQ ID NO:1 of the sequence listing.

Analysis of a population of plants having the wild-type phenotype and producing grains containing a normal embryo has made it possible to identify at least two variants of the isum2A gene. One of the variants of the gene is characterized by the substitution of the G nucleotide located at position 2234 of the sequence SEQ ID NO:1 with a C nucleotide. This nucleotide substitution leads to the substitution of the amino acid G (glycine) at position 89 of the ISUM2A polypeptide sequence SEQ ID NO:5 with an amino acid R (asparagine) which is present in the sequence of the variant ISUM2A polypeptide, SEQ ID N°6.

Thus, a first subject of the invention consists of a nucleic acid comprising a polynucleotide encoding an ISUM2A polypeptide chosen from the sequences having at least 95% amino acid identity with the sequences SEQ ID NO:5 and SEQ ID NO:6, or encoding a fragment of an ISUM2A polypeptide.

The invention also relates to a nucleic acid of sequence complementary to the nucleic acid as defined above.

According to the invention, any conventional molecular biology, microbiology and recombinant DNA techniques known to those skilled in the art may be used. Such techniques are described, for example, by Sambrook et al. (1989), Glover (1985), Gait (1984), Hames and Higgins (1984), Berbal (1984) and Ausubel et al. (1994).

Preferably, any nucleic acid and any polypeptide according to the invention is in an isolated or purified form.

For the purpose of the present invention, the term “isolated” denotes a biological material which has been removed from its original environment (the environment in which it is naturally located). For example, a polynucleotide present in the natural state in a plant is not isolated. The same polynucleotide separated from the adjacent nucleic acids into which it is naturally inserted in the genome of the plant is isolated. Such a polynucleotide may be included in a vector, and/or such a polynucleotide may be included in a composition, and nevertheless remain in the isolated state due to the fact that the vector or the composition does not constitute its natural environment.

The term “purified” does not require the material to be present in a form of absolute purity, excluding the presence of other compounds. It is rather a relative definition. A polynucleotide or a polypeptide is in the purified state after purification of the starting material or of the natural material by at least one order of magnitude, preferably 2 or 3, and preferentially four or five orders of magnitude.

For the purposes of the present invention, the expression “nucleotide sequence” may be used to denote equally a polynucleotide or a nucleic acid. The expression “nucleotide sequence” encompasses the genetic material itself and is not therefore restricted to the information concerning its sequence.

The terms “nucleic acid”, “polynucleotide”, “oligonucleotide” or alternatively “nucleotidique sequence” encompass RNA, DNA or cDNA sequences, or else RNA/DNA hybrid sequences, of more than one nucleotide, equally in single-stranded form or in the form of a duplex.

The term “nucleotide” denotes both natural nucleotides (A, T, G, C) and modified nucleotides which comprise at least one modification including, without limitation, (i) a purine analog, (ii) a pyrimidine analog, or (iii) a sugar analog, such modified nucleotides being described, for example, in PCT application No. WO 95/04064.

For the purposes of the present invention, a first polynucleotide is considered to be “complementary” to a second polynucleotide when each base of the first nucleotide is paired with the complementary base of the second polynucleotide, the orientation of which is inverted. The complementary bases are A and T (or A and U), and C and G.

According to the invention, a first nucleic acid having at least 95% identity with a reference second nucleic acid would have at least 95%, preferably at least 96%, 97%, 98%, 98.5%, 99%, 99.1%, 99.2%, 99.3%, 99.4%, 99.5%, 99.6%, 99.7%, 99.8% or 99.9% nucleotide identity with this reference second polynucleotide, the percentage identity between two sequences being determined as described below.

For the purpose of the present invention, the “percentage identity” between two nucleotide or amino acid sequences can be determined by comparing two optimally aligned sequences, through a window of comparison. The part of the nucleotide or polypeptide sequence in the window of comparison may thus comprise additions or deletions (for example “gaps”) compared to the reference sequence (which does not comprise these additions or these deletions) so as to obtain an optimal alignment of the two sequences. The percentage is calculated by dividing the number of positions at which an identical nucleic acid base or amino acid residue is observed for the two compared (nucleic acid or amino acid) sequences by the total number of positions in the window of comparison and multiplying the quotient by one hundred.

The optimal alignment of the sequences for the comparison may be carried out on a computer using known algorithms. Preferably, the percentage sequence identity is determined by means of the BLAST software (BLAST version 2.06 from September 1998), using exclusively the default parameters.

A nucleic acid having at least 95% nucleotide identity with a nucleic acid according to the invention encompasses the “variants” of a nucleic acid according to the invention. The term “variant” of a nucleic acid according to the invention is intended to mean a nucleic acid which differs from the reference nucleic acid by one or more substitutions, additions or deletions of a nucleotide, compared to the reference nucleic acid. A variant of a nucleic acid according to the invention may be of natural origin, such as an allelic variant which exists naturally. Such a variant nucleic acid may also be an unnatural nucleic acid obtained, for example, by mutagenesis techniques.

In general, the differences between the reference nucleic acid and the “variant” nucleic acid are small such that the reference nucleic acid and the variant nucleic acid are nucleotide sequences which are very similar and, in many regions, identical. The nucleotide modifications present in a variant nucleic acid may be silent, which means that they do not affect the amino acid sequence which may be encoded by this variant nucleic acid. The nucleotide modifications in the variant nucleic acid may also result in substitutions, additions or deletions of one or more amino acids in the sequence of the polypeptide which may be encoded by this variant nucleic acid.

In a preferred embodiment of the invention, a variant nucleic acid comprising an open reading frame encodes a polypeptide which conserves the same biological function or the same biological activity as the polypeptide encoded by the reference nucleic acid. A variant nucleic acid according to the invention which comprises an open reading frame encodes a polypeptide which preferably conserves the ability to be recognized by antibodies directed against the polypeptide encoded by the reference nucleic acid.

The nucleic acids of the genes orthologous to ISUM2 included in the genome of plants other than maize, and having a nucleotide identity of at least 95% with a nucleic acid encoding the ISUM2A polypeptide, are part of the “variants” of a nucleic acid encoding the ISUM2A polypeptide.

The “fragment” of a nucleic acid according to the invention is intended to mean a nucleotide sequence which is shorter compared to the reference nucleic acid, the nucleic acid fragment having a nucleotide sequence identical to the nucleotide sequence of the reference nucleic acid over the common portion. Such fragments of a nucleic acid according to the invention have at least 12, 15, 18, 20, 25, 30, 35, 40, 45, 50, 60, 70, 80, 90, 100, 150, 200, 300, 400, 500, 1000, 2000 or 3000 consecutive nucleotides of the reference nucleic acid, the maximum length in nucleotides of a fragment of a nucleic acid according to the invention being, of course, limited by the maximum length in nucleotides of the reference nucleic acid.

The term “fragment” of an ISUM2A polypeptide according to the invention is intended to mean a polypeptide fragment which is shorter compared to the reference polypeptide, the polypeptide fragment having an amino acid sequence identical to the amino acid sequence of the reference polypeptide over the common portion. Such fragments of an ISUM2A polypeptide according to the invention have at least 5, 10, 15, 20, 25, 30, 40, 50, 60, 70, 80, 90, 100, 120, 130, 135 or 140 consecutive amino acids of a reference ISUM2A polypeptide.

Isum2A Genomic Nucleic Acids

As indicated above, two allelic variants of the genomic nucleic acid of the isum2A gene have been characterized according to the invention, the two variant genomic nucleic acids being respectively referenced as the nucleotide sequences SEQ ID NO:1 and SEQ ID NO:2 of the sequence listing. Consequently, the present invention provides a nucleic acid encoding an ISUM2A polypeptide, or encoding a fragment of this polypeptide, said nucleic acid comprising a polynucleotide having at least 95% nucleotide identity with a nucleotide sequence chosen from SEQ ID NO:1 and SEQ ID NO:2, or with a fragment of one of the sequences SEQ ID NO:1 and SEQ ID NO:2. The invention also provides a nucleic acid of sequence complementary to the nucleic acid as defined above.

Another subject of the invention is a nucleic acid consisting of a polynucleotide having at least 95% nucleotide identity with a sequence chosen from the sequences SEQ ID NO:1 and SEQ ID NO:2, or with a fragment of one of the sequences SEQ ID NO: 1 or SEQ ID NO:2, or a nucleic acid of complementary sequence.

The invention also relates to a nucleic acid comprising at least 12, preferably at least 15, and entirely preferably at least 20, consecutive nucleotides of the nucleic acid of sequence SEQ ID NO:1 or SEQ ID NO:2, it being understood that such a nucleic acid encompasses in its definition the “fragments” of a nucleic acid according to the invention as defined in the present description.

The isum2A gene, defined by the sequence SEQ ID NO:1, comprises, from the 5′ end to the 3′ end, respectively:
(a) a noncoding sequence potentially carrying regulatory elements for transcription and/or translation of this gene, located upstream of the first exon, from the nucleotide at position 1 to the nucleotide at position 1812 of the sequence SEQ ID NO:1;
(b) a “coding” region which comprises the three exons and the two introns of the isum2A gene, this coding region being located from the nucleotide at position 1813 to the nucleotide at position 4074 of the sequence SEQ ID NO:1; and
(c) a noncoding region located downstream of the coding region, from the nucleotide at position 4075 to the nucleotide at position 5620 of the sequence SEQ ID NO:1.

The sequence SEQ ID NO:2, which illustrates a variant of the isum2A gene, does not contain the entire open reading frame encoding an ISUM2A polypeptide. The sequence SEQ ID NO:2 comprises a portion located on the 3′ side of exon No. 1, and also all of exons No. 2 and No. 3. The nucleotide at position 1 of the sequence SEQ ID NO:2 corresponds to the nucleotide at position 2063 of the sequence SEQ ID NO: 1. The nucleotide at position 2004 of the sequence SEQ ID NO:2 corresponds to the nucleotide at position 4062 of the sequence SEQ ID NO: 1.

However, knowledge of the sequence SEQ ID NO:2 by those skilled in the art and comparison thereof with sequence SEQ ID NO:1, makes it possible to directly obtain the entire coding sequence of the isum2A gene defined by the sequence SEQ ID NO:2, for example by completing the missing bases of exon No. 1 of the sequence SEQ ID NO:2 with the corresponding bases of exon No. 1 of the sequence SEQ ID NO:1, using as a basis the corresponding positions given above and also in Table 1 below.

Similarly, those skilled in the art can obtain the nucleic acid of the isum2A gene, for example by isolating, from the information of the sequence SEQ ID NO:1, the equivalent nucleotide regions or else by producing a hybrid nucleic acid obtained by fusing the nucleic acids of sequences SEQ ID NO:1 and SEQ ID NO:2 on the basis of the corresponding positions given above and also in Table 1 below.

The structural characteristics of the three introns and of the two exons of the ISUM2A gene are given in detail in Table 1 below.

TABLE 1
Sequences of the exons of the Isum2A gene

| | Position of the nucleotide | | Position of the nucleotide | |
|---|---|---|---|---|
| Exon | in the 5′ position on | | in the 3′ position on | |
| No. | SEQ ID NO: 1 | SEQ ID NO: 2 | SEQ ID NO: 1 | SEQ ID NO: 2 |
| 1 | 1813 | <1 | 2099 | 37 |
| 2 | 2207 | 146 | 2323 | 262 |
| 3 | 3646 | 1588 | 4074 | >2004 |

The invention also relates to a nucleic acid comprising at least 12 consecutive nucleotides of an exonic polynucleotide of the Isum2A gene, such as the polynucleotides 1 to 3 described in Table 1 above, which are included in the nucleic acid of sequence SEQ ID NO:1 and SEQ ID NO:2.

Such a nucleic acid encodes at least part of the polypeptide encoded by the Isum2A gene and can in particular be inserted into a recombinant vector intended for the expression of the corresponding translation product in a host cell or in a plant transformed with this recombinant vector.

Such a nucleic acid may also be used for synthesizing nucleotide probes and primers intended for the detection or for the amplification of nucleotide sequences included in the Isum2A gene in a sample, where appropriate, of sequences of the Isum2A gene carrying one or more mutations, preferably one or more mutations which are such that they modify the phenotype of a plant carrying such a mutated Isum2A gene, by causing the production of grains altered in germ development.

TABLE 2
Sequences of the introns of the Isum2A gene

| | |
|---|---|
| Position of the | Position of the |
| nucleotide in the | nucleotide in the |
| 5′ position on | 3′ position on |

| | SEQ ID | SEQ ID | SEQ ID | SEQ ID |
|---|---|---|---|---|
| Intron No. | NO: 1 | NO: 2 | NO: 1 | NO: 2 |
| 1 | 2100 | 38 | 2206 | 145 |
| 2 | 2324 | 263 | 3645 | 1587 |

The invention also relates to a nucleic acid comprising at least 12 consecutive nucleotides of an intronic polynucleotide of the Isum2A gene, such as the polynucleotides 1 and 2 described in Table 2 above, which are included in the nucleic acid of sequence SEQ ID NO:1 and SEQ ID NO:2.

Such a nucleic acid may be used as an oligonucleotide probe or primer for detecting the presence of at least one copy of the Isum2A gene in a sample, or else for amplifying a given target sequence in the Isum2A gene.

Such a nucleic acid may also be used for amplifying a given target sequence in the isum2A gene or inhibiting it by a sense or cosuppression approach, or using double-stranded RNA (Wassenegger et al. 1996; Kooter et al. 1999) for interference. Such a nucleic acid may also be used for searching for functional allelic variants of the ISUM2 gene, which may be used in a method for selecting plants with an embryo modified in terms of size and/or development.

The region of the genomic Isum2A gene essentially restricted to the “coding” region comprising the 3 exons and the 2 introns is defined as the sequence beginning at the nucleotide at position 1813 and ending at the nucleotide at position 4074 of the sequence SEQ ID NO:1.

Part of exon No. 1, all of exons No. 2 and 3 and also the two introns of a variant of the isum2A gene are included in the sequence ranging from the nucleotide at position 1 to the nucleotide at position 2004 of the sequence SEQ ID NO:2.

It is specified that, over their common region, the sequences SEQ ID NO:1 and SEQ ID NO:2 have a percentage nucleotide identity of greater than 95%, this percentage in fact being greater than 99%.

A subject of the invention is also a nucleic acid comprising a polynucleotide having at least 95% nucleotide identity with the nucleotide sequence beginning at the nucleotide at position 1813 and ending at the nucleotide at position 4074 of the sequence SEQ ID NO:1, and also a nucleic acid of complementary sequence.

The invention also relates to a nucleic acid having at least 95% nucleotide identity with the nucleotide sequence beginning at the nucleotide at position 1813 and ending at the nucleotide at position 4074 of the sequence SEQ ID NO:1, and also a nucleic acid of complementary sequence.

A subject of the invention is also a nucleic acid comprising the nucleotide sequence beginning at the nucleotide in position 1813 and ending at the nucleotide at position 4074 of the sequence SEQ ID NO:1 or a nucleic acid of complementary sequence.

The invention also relates to a nucleic acid consisting of the nucleotide sequence beginning at the nucleotide at position 1813 and ending at the nucleotide at position 4074 of the sequence SEQ ID NO:1 or a nucleic acid of complementary sequence.

Another subject of the invention consists of a nucleic acid characterized in that it comprises one of the following nucleotide sequences:
(a) the sequence ranging from the nucleotide at position 1 to the nucleotide at position 1812 of the sequence SEQ ID NO:1, or a nucleic acid of complementary sequence;
(b) the sequence ranging from the nucleotide at position 1813 to the nucleotide at position 2099 of the sequence SEQ ID NO:1, or a nucleic acid of complementary sequence;
(c) the sequence ranging from the nucleotide at position 2100 to the nucleotide at position 2206 of the sequence SEQ ID NO:1, or a nucleic acid of complementary sequence;
(d) the sequence ranging from the nucleotide at position 2207 to the nucleotide at position 2323 of the sequence SEQ ID NO:1, or a nucleic acid of complementary sequence;
(e) the sequence ranging from the nucleotide at position 2324 to the nucleotide at position 3645 of the sequence SEQ ID NO:1, or a nucleic acid of complementary sequence;
(f) the sequence ranging from the nucleotide at position 3646 to the nucleotide at position 4074 of the sequence SEQ ID NO:1, or a nucleic acid of complementary sequence; and
(g) the sequence ranging from the nucleotide at position 4075 to the nucleotide as position 5620 of the sequence SEQ ID NO:1, or a nucleic acid of complementary sequence.

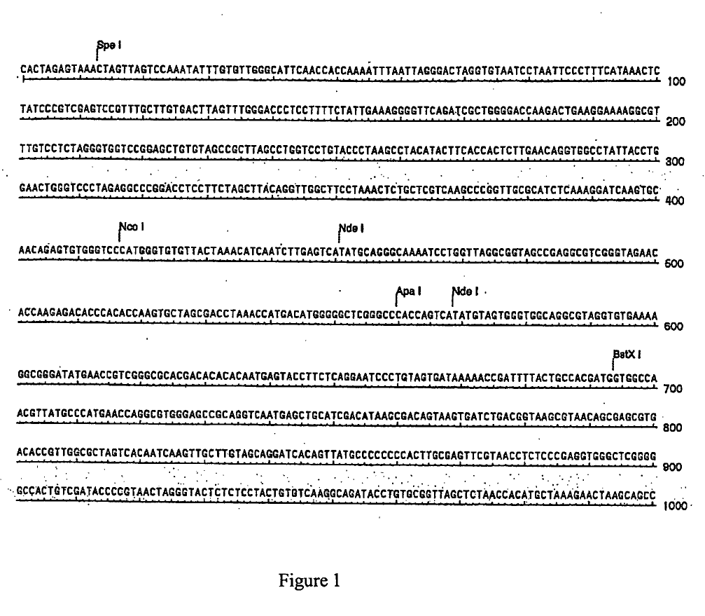

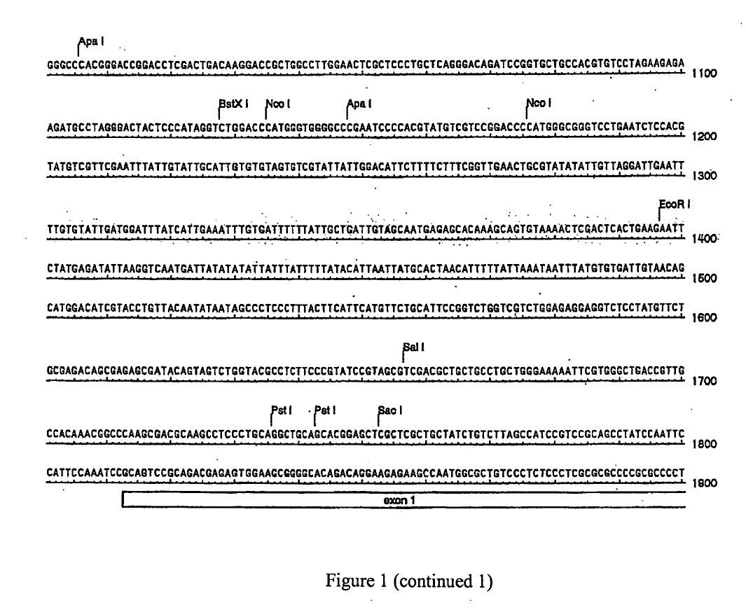

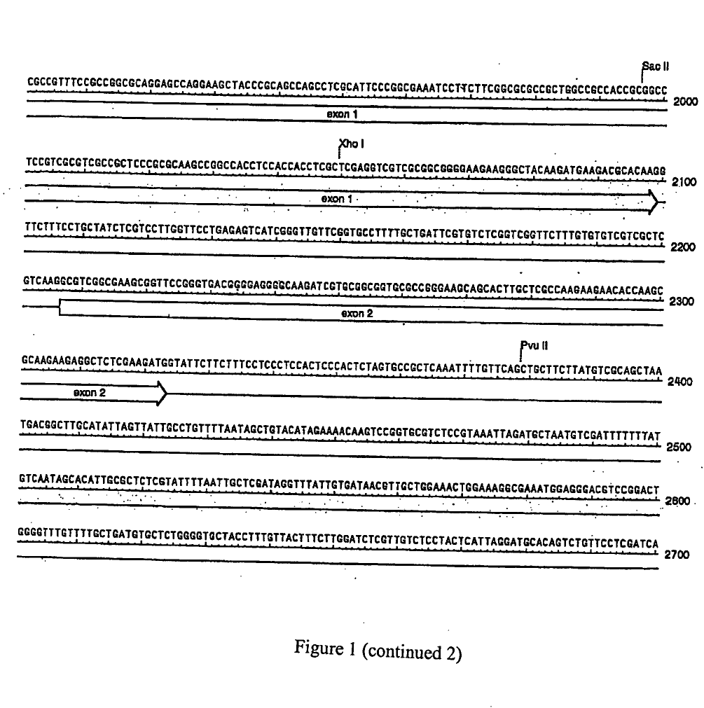

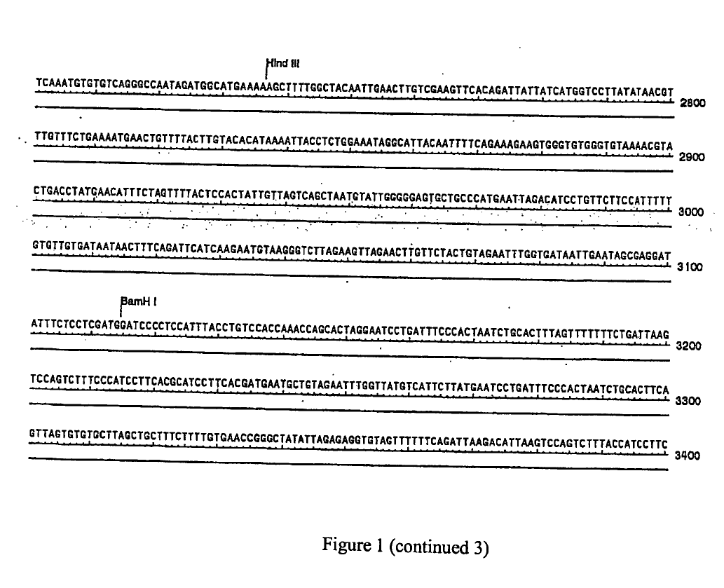

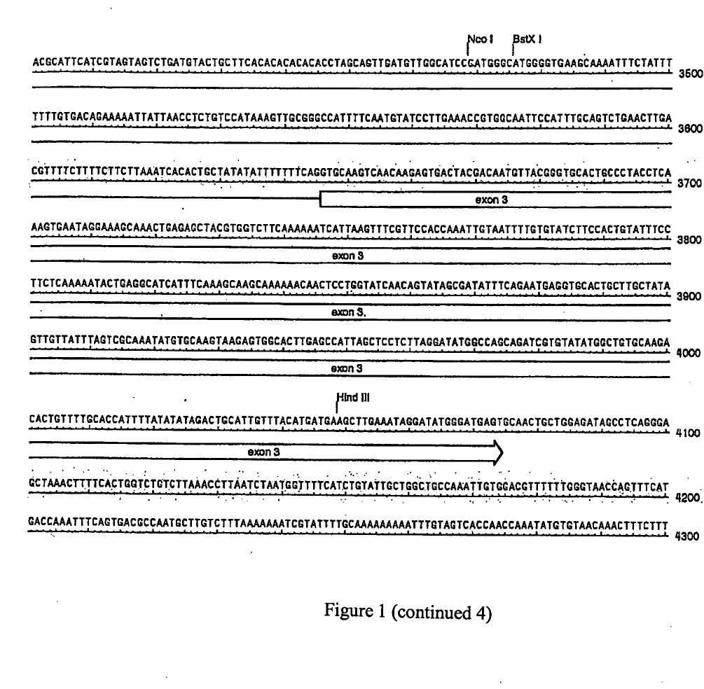

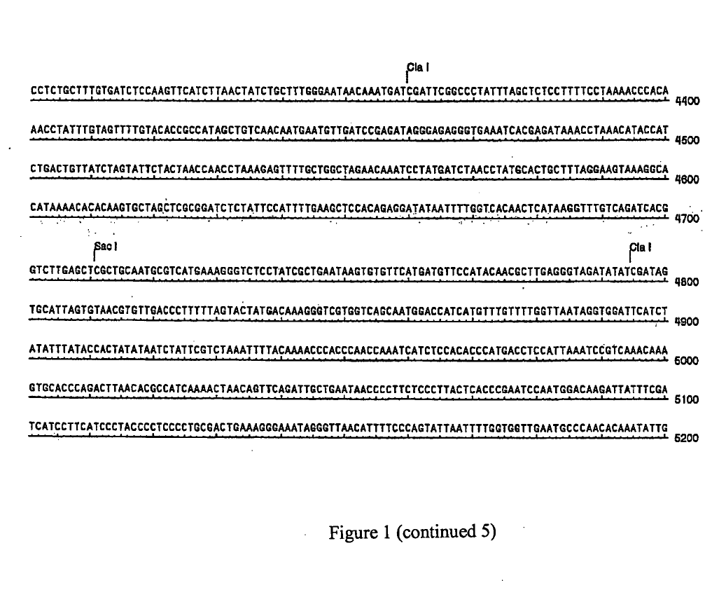

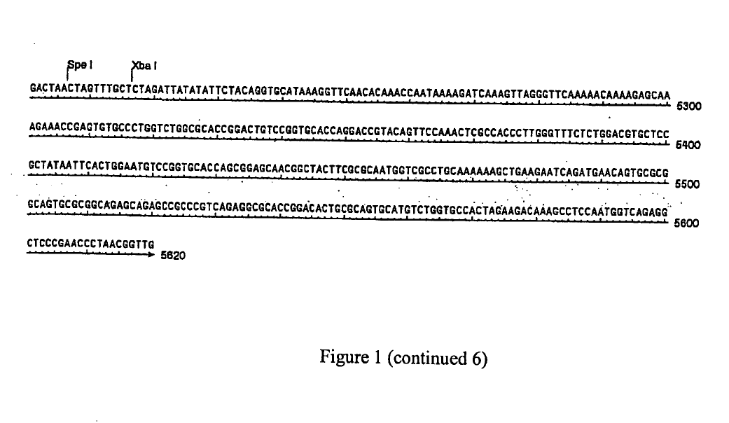

The nucleic acid of sequence SEQ ID NO:1 is represented in FIG. 1, in which details are also given of the positions of the various exons and introns of the Isum2A gene.

Products of Transcription of the Isum2A Gene

It has been shown, according to the invention, that the Isum2A gene is transcribed in the form of a messenger RNA. This messenger RNA comprises an open reading frame encoding the ISUM2A protein.

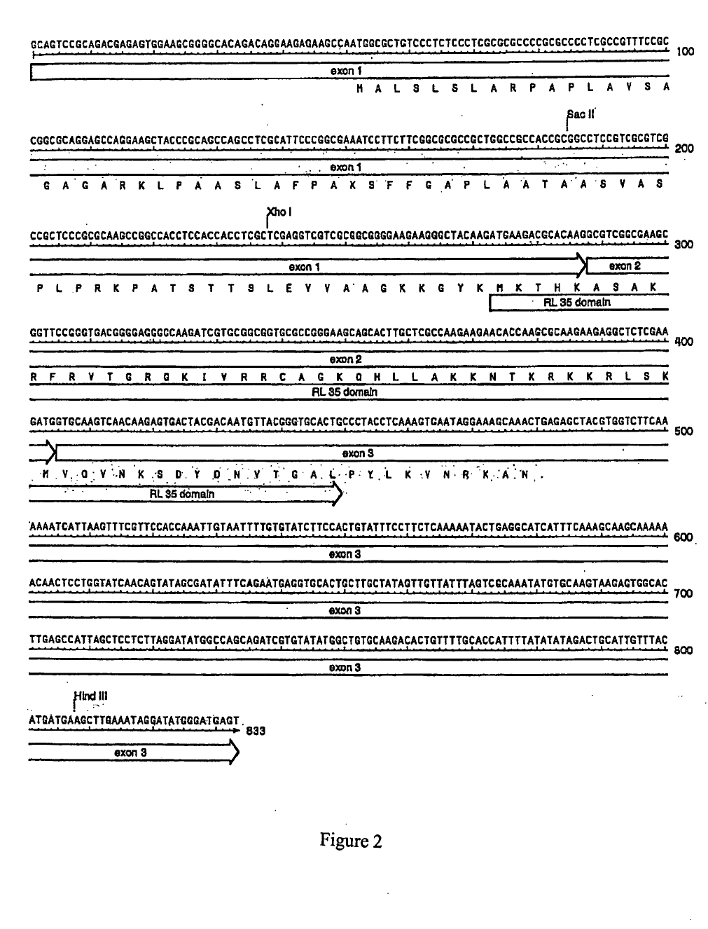

The part of the cDNA of the Isum2A gene comprising the open reading frame encoding the ISUM2A polypeptide of sequence SEQ ID NO:5 is 833 nucleotides in length and is referenced as the sequence SEQ ID NO:3 of the sequence listing. The cDNA of sequence SEQ ID NO:3 was derived from the wild-type plant variant denoted HD5×HD7. This cDNA is represented in FIG. 2.

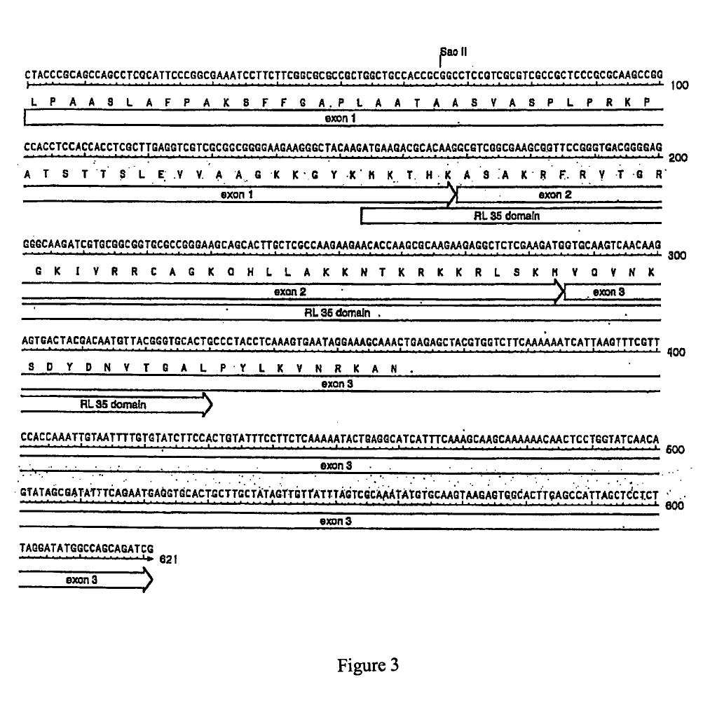

The part of the cDNA of the Isum2A gene comprising part of the open reading frame encoding the ISUM2A polypeptide of sequence SEQ ID NO:6 is 621 nucleotides in length and is referenced as the sequence SEQ ID NO:4 of the sequence listing. The cDNA of sequence SEQ ID NO:4 was derived from the wild-type plant variant denoted A188. This partial cDNA is represented in FIG. 3.

The nucleic acids of sequences SEQ ID NO:3 and SEQ ID NO:4 exhibit similarities with an EST sequence referenced in the GENBANK database under the accession number A1001298 and denoted “ISUM2”. For this reason, the newly discovered gene according to the invention has been given the name “isum2A”. The sequence of the EST No. AI001298 has a degree of nucleotide identity of 94% and 95% with the sequences SEQ ID NO:3 and SEQ ID NO:4, respectively. No open reading frame is described for this EST.

The nucleic acids of sequences SEQ ID NO:3 and SEQ ID NO:4 also exhibit similarities with an EST sequence referenced in the GENBANK database under the accession number AI374506. This sequence was obtained from the same clone “MEST6-D3” as the sequence AI001298. The sequence of the EST No. AI374506 has a degree of nucleotide identity of 92% and 98% with the sequences SEQ ID NO:3 and SEQ ID NO:4, respectively. No open reading frame is described for this EST.

Another subject of the invention consists of a nucleic acid comprising a polynucleotide encoding an ISUM2A polypeptide and having at least 99% nucleotide identity with the nucleotide sequence SEQ ID NO:3 or SEQ ID NO:4, or with a fragment of this nucleotide sequence, and also a nucleic acid of sequence complementary to these nucleic acids.

The invention also relates to a nucleic acid encoding an ISUM2A polypeptide and having at least 99% nucleotide identity with the nucleotide sequence SEQ ID NO:3 or SEQ ID NO:4, or a fragment of this nucleotide sequence, and also a nucleic acid of sequence complementary to these nucleic acids.

A subject of the invention is also a nucleic acid characterized in that it comprises the nucleotide sequence SEQ ID NO:3 or SEQ ID NO: 4 or a nucleic acid of complementary sequence. Also part of the invention is a nucleic acid consisting of the nucleotide sequence SEQ ID NO:3 or SEQ ID NO:4, or a nucleic acid of complementary sequence.

The Isum2A gene encodes a polypeptide 143 amino acids in length, two allelic variants of which have been identified according to the invention, respectively the variant polypeptides of sequence SEQ ID NO:5 and SEQ ID NO:6, which have a degree of identity of more than 99% with one another. Consequently, the invention also relates to a nucleic acid encoding a polypeptide having at least 95% amino acid identity with the sequence SEQ ID NO:5 or SEQ ID NO:6. The invention also relates to a nucleic acid characterized in that it encodes the polypeptide of sequence SEQ ID NO:5 or SEQ ID NO:6.

Also part of the invention is a nucleic acid encoding a “fragment” of a polypeptide having at least 95% nucleotide identity with a polypeptide of amino acid sequence SEQ ID NO:5 or SEQ ID NO:6.

The invention also relates to a nucleic acid encoding a fragment of a polypeptide of amino acid sequence SEQ ID NO:5 or SEQ ID NO:6. A polypeptide having at least 95% amino acid identity with a reference polypeptide comprises at least 96%, 97%, 98%, 99%, 99.1%, 99.2%, 99.3%, 99.4%, 99.5%, 99.6%, 99.7%, 99.8% or 99.9% amino acid identity with the reference polypeptide.

The “variant” polypeptides are encompassed in the definition of a polypeptide having at least 95% amino acid identity with a reference polypeptide according to the invention. The “variant” of a polypeptide according to the invention is intended to mean a polypeptide the amino acid sequence of which comprises one or more substitutions, additions or deletions of at least one amino acid residue, compared to the amino acid sequence of the reference polypeptide, it being understood that the amino acid substitutions may be conservative or nonconservative in nature.

A variant of a reference polypeptide according to the invention consists of a polypeptide which conserves the biological function or the biological activity of the reference polypeptide and/or which is recognized by antibodies directed against the reference polypeptide. These polypeptide variants may result from allelic variations characterized by differences in the nucleotide sequences of the gene encoding these polypeptides. Such polypeptide variants may also result from alternative splicing or from post-translation modifications.

The term “fragment” of a reference polypeptide according to the invention is intended to mean a polypeptide having at least 10, 15, 20, 25, 30, 40, 50, 60, 70, 80, 90, 100, 110, 120, 130, 135 or 140 consecutive amino acids of a polypeptide as defined in the present description.

Probes and Primers According to the Invention

The nucleic acids according to the invention, and in particular the nucleotide sequences SEQ ID NO:1 to SEQ ID NO:4, their fragments of at least 12 nucleotides, the sequences having at least 95% nucleotide identity with at least part of the sequences SEQ ID NO:1 to SEQ ID NO:4, and also the nucleic acids of complementary sequence, are useful for detecting the presence of at least one copy of a nucleotide sequence of the Isum2A gene or else of a fragment or else of an allelic variant of this sequence, in a sample.

Also part of the invention are the nucleotide probes and primers which hybridize, under high stringency hybridization conditions, with a nucleic acid chosen from the sequences SEQ ID NO:1 to SEQ ID NO:4. The hybridization conditions below are used for the hybridization of a nucleic acid, probe or primer, 20 bases in length. The level and the specificity of hybridization depend on various parameters, such as:
(a) the purity of the preparation of the nucleic acid to which the probe or the primer must hybridize;
(b) the base composition of the probe or of the primer, G-C base pairs having greater thermal stability than A-T or A-U base pairs;
(c) the length of the sequence of bases which are homologous between the probe or the primer and the nucleic acid;
(d) the ionic strength: the hybridization rate increases with an increase in ionic strength and the duration of the incubation time;
(e) the incubation temperature;
(f) the concentration of the nucleic acid to which the probe or the primer must hybridize;
(g) the presence of denaturing agents such as agents which promote cleavage of hydrogen bonds, for instance formamide or urea, which increase the stringency of the hybridization;
(h) the incubation time, the hybridization rate increasing with the duration of incubation;
(i) the presence of volume excluders, such as dextran or dextran sulfate, which increase the rate of hybridization due to the fact that they increase the effective concentrations of the probe or of the primer and of the nucleic acid which must hybridize, within the preparation.;

The parameters defining the conditions of stringency depend on the temperature at which 50% of the paired strands separate (Tm).

For sequences comprising more than 360 bases, Tm is defined by the relationship:

Tm=81.5+0.41 (% G+C)+16.6 Log(cation concentration)−0.63 (% formamide)−(600/number of bases) (Sambrook et al., (1989), pages 9.54-9.62). For sequences less than 30 bases in length, Tm is defined by the relationship: Tm=4(G+C)+2(A+T). Under suitable conditions of stringency, under which nonspecific sequences do not hybridize, the hybridization temperature is approximately from 5 to 30° C., preferably from 5 to 10° C., below Tm.

The expression “high stringency hybridization conditions” according to the invention is intended to mean hybridization conditions such that the procedure is carried out at a hybridization temperature of 5° C. below the Tm.

The hybridization conditions described above can be adjusted as a function of the length and of the base composition of the nucleic acid for which hybridization is sought or of the type of labeling chosen, according to techniques known to those skilled in the art. Suitable hybridization conditions may, for example, be adjusted according to the teaching contained in the work by Hames and Higgins (1985) or else in the work by Ausubel et al. (1989).

By way of illustration, the hybridization conditions used for a nucleic acid 200 bases in length are as follows:

Prehybridization

Conditions: same as for the hybridization.

Duration: overnight.

Hybridization

Conditions: 5×SSPE (0.9 M NaCl, 50 mM sodium phosphate, pH 7.7, 5 mM EDTA)

5× Denhardt's (0.2% PVP, 0.2% Ficoll, 0.2% BSA)

100 μg/ml salmon sperm DNA

0.1% SDS

Duration: overnight.

Washes

2×SSC, 0.1% SDS 10 min 65° C.

1×SSC, 0.1% SDS 10 min 65° C.

0.5×SSC, 0.1% SDS 10 min 65° C.

0.1×SSC, 0.1% SDS 10 min 65° C.

The nucleotide probes or primers according to the invention comprise at least 12 consecutive nucleotides of a nucleic acid according to the invention, in particular of a nucleic acid of sequences SEQ ID NOS:1 to 4 or of the sequence complementary thereto, of a nucleic acid having 95% nucleotide identity with a sequence chosen from the sequences SEQ ID NOS:1 to 4 or of the sequence complementary thereto, or else of a nucleic acid which hybridizes, under high stringency hybridization conditions, with a sequence chosen from the sequences SEQ ID NOS:1 to 4 or of the sequence complementary thereto.

Preferably, nucleotide probes or primers according to the invention will have a length of at least 12, 15, 18, 20, 25, 30, 35, 40, 45, 50, 60, 100, 150, 200, 300, 400, 500, 1000, 2000 or 3000 consecutive nucleotides of a nucleic acid according to the invention. Alternatively, a nucleotide probe or primer according to the invention will consist of and/or will comprise the fragments with a length of 12, 15, 18, 20, 25, 30, 35, 40, 45, 50, 60, 100, 150, 200, 300, 400, 500, 1000, 2000 or 3000 consecutive nucleotides of a nucleic acid according to the invention.

Examples of primers or pairs of primers for amplifying a nucleic acid fragment of the Isum2A gene of sequence SEQ ID NO:1 or SEQ ID NO:2 are, for example, the primers SEQ ID NOS: 10 to 13 and 17 to 23. The use of the primers of sequences SEQ ID NOS :10 to 13 and 17 to 23 in amplification reactions is described in the examples.

A nucleotide primer or probe according to the invention can be prepared by any suitable method well known to those skilled in the art, including by cloning and the action of restriction enzymes, or else by direct chemical synthesis according to techniques such as the phosphodiester method of Narang et al. (1979) or of Brown et al. (1979), the diethylphosphroamidite method of Beaucage et al. (1980) or else the solid support technique described in European patent No. EP 0 707 592. Each of the nucleic acids according to the invention, including the oligonucleotide probes and primers described above, can be labeled, if desired, by incorporating a molecule which is detectable, i.e. a label which is detectable, by spectroscopic, photochemical, biochemical, immunochemical or else chemical means.

For example, such labels can consist of radioactive isotopes (³²P ³H, 35S), fluorescent molecules (5-bromodeoxyuridine, fluorescein, acetylaminofluorene) or else ligands such as biotin.

The labeling of the probes is preferably carried out by incorporation of labeled molecules into the polynucleotides by primary extension, or else by addition to the 5′ or 3′ ends. Examples of nonradioactive labeling of nucleic acid fragments are described in particular in French patent No. FR 78 10 975 or else in the articles by Urdea et al. (1988) or Sanchez Pescador et al. (1988). Advantageously, the probes according to the invention may have structural characteristics such that they allow amplification of the signal, such as the probes described by Urdea et al. (1991) or else European patent No. EP 0 225 807 (Chiron).

The oligonucleotide probes according to the invention can be used in particular in Southern-type hybridizations to the genomic DNA of the Isum2A gene or else in hybridizations to the messenger RNA of this gene when the expression of the corresponding transcript is sought in a sample. The probes according to the invention can also be used for detecting PCR amplification products or else for detecting mismatches.

Nucleotide probes or primers according to the invention can be immobilized on a solid support. Such solid supports are well known to those skilled in the art and comprise surfaces of microtitration plate wells, polystyrene beads, magnetic beads, nitrocellulose strips or else microparticles such as latex particles.

Consequently, a subject of the invention is also a nucleic acid which can be used as a nucleotide probe or primer, characterized in that it comprises at least 12 consecutive nucleotides of a nucleic acid as defined above, in particular of a nucleic acid of nucleotide sequences SEQ ID NO:1 to SEQ ID NO:4.

The invention also relates to a nucleic acid which can be used as a nucleotide probe or primer, characterized in that it consists of a polynucleotide of at least 12 consecutive nucleotides of a nucleic acid according to the invention, most preferably of a nucleic acid of sequences chosen from the nucleotide sequences SEQ ID NO:1 to SEQ ID NO:4.

As described above, such a nucleic acid may also be characterized in that it is labeled with a detectable molecule.

A nucleic acid which can be used as a nucleotide probe or primer for detecting or amplifying a genomic, mRNA or cDNA sequence of the Isum2A gene can also be characterized in that it is chosen from the following sequences:
(a) the nucleotide sequences which hybridize, under high stringency hybridization conditions, with a nucleic acid of sequence SEQ ID NO:1 or SEQ ID NO:2; and
(b) the sequences comprising at least 12 consecutive nucleotides of a nucleic acid of sequence SEQ ID NO:1 or SEQ ID NO:2.

The present invention also relates to a method for detecting the presence of a nucleic acid of the Isum2A gene in a sample, said method comprising the steps of:
1) bringing a nucleotide probe or a plurality of nucleotide probes according to the invention into contact with the test sample;
2) detecting the complex possibly formed between the probe(s) and the nucleic acid present in the sample.

According to a particular embodiment of the method of detection according to the invention, the oligonucleotide probe(s) is (are) immobilized on a support. According to another aspect, the oligonucleotide probes comprise a detectable label.

The invention also relates to a pack or kit for detecting the presence of a nucleic acid according to the invention in a sample, said pack comprising:
(a) one or more nucleotide probes as defined above;
(b) where appropriate, the reagents required for the hybridization reaction.

According to a first aspect, the detection pack or kit is characterized in that the probe(s) is (are) immobilized on a support. According to a second aspect, the detection pack or kit is characterized in that the oligonucleotide probes comprise a detectable label.

According to a particular embodiment of the detection kit described above, such a kit will comprise a plurality of oligonucleotide probes in accordance with the invention, which will be used for detecting target sequences of interest of the Isum2A gene, or alternatively detecting mutations in the coding regions or the noncoding regions of the Isum2A gene, more particularly nucleic acids of sequence SEQ ID NO:1 to SEQ ID NO:4 or the nucleic acids of complementary sequence.

For the purpose of the invention, the term “target sequence” is intended to mean a nucleotide sequence comprised in a nucleic acid, said nucleotide sequence hybridizing, under the hybridization conditions specified in the description, with a nucleotide probe or primer of the invention.

A target sequence may be, for example, a sequence comprised in a regulatory nucleic acid for the Isum2A gene or else a sequence comprised in a genomic coding region or a coding region of the cDNA of this gene.

The nucleotide primers according to the invention may be used for amplifying any nucleotide fragment (gDNA, cDNA, mRNA) of the Isum2A gene, and more particularly all or part of a nucleic acid of sequence SEQ ID NO:1 to SEQ ID NO:4, or else a fragment or a variant of these sequences.

Another subject of the invention concerns a method for amplifying a nucleic acid according to the invention, and more particularly a nucleic acid of sequence SEQ ID NO:1 to SEQ ID NO:4 or a fragment or an allelic variant thereof, contained in a sample, said method comprising the steps of:
(a) bringing the sample in which the presence of the target nucleic acid is suspected into contact with a pair of nucleotide primers the hybridization position of which is located, respectively, on the 5′ side and on the 3′ side of the region of the target nucleic acid of the Isum2A gene whose amplification is desired, in the presence of the reagents required for the amplification reaction; and
(b) detecting the nucleic acid possibly amplified.

The nucleotide primers described hereinafter may be advantageously used to implement the method of amplification as defined above.

A subject of the invention is also a pack or kit for amplifying a nucleic acid according to the invention, and more particularly all or part of a nucleic acid of sequences SEQ ID NOS:1 to 4, said pack or kit comprising:
(a) a pair of nucleotide primers in accordance with the invention, the hybridization position of which is located, respectively, on the 5′ side and on the 3′ side of the target nucleic acid of the Isum2A gene whose amplification is desired;
(b) where appropriate, the reagents required for the amplification reaction.

Such an amplification pack or kit will advantageously comprise at least one pair of nucleotide primers as described above.

According to a preferred embodiment, primers according to the invention comprise all or part of a polynucleotide chosen from the nucleotide sequences SEQ ID NOS:10 to 13 and 17 to 23.

Nucleic Acid Constructs According to the Invention

The isolation and characterization of the isum2A gene according to the invention and the demonstration of the need for expression of the isum2A gene at a detectable level in the plant in order to obtain normal development of the embryo of the grain, after pollenation, have enabled the inventors to prepare nucleic acid constructs which allow the expression of at least one functional copy of the isum2A gene in a cellular host, particularly in a plant cell transformed with such nucleic acid constructs.

Preferably, said cell carries at least one nonfunctional isum2A allele. More preferably, said cell carries the two nonfunctional isum2A alleles.

According to the invention, nucleic acid constructs have been developed which make it possible to obtain controlled expression of at least one functional copy of a nucleic acid encoding an ISUM2A polypeptide in a cellular host, particularly in a plant cell.

Consequently, a subject of the invention is also a nucleic acid comprising a polynucleotide encoding an ISUM2A polypeptide as defined in the present description, or encoding a fragment of this polypeptide, said nucleic acid also comprising an expression control sequence capable of regulating the transcription or the translation of the polynucleotide encoding the ISUM2A polypeptide, or the fragment of this polypeptide.

Also part of the invention is the nucleic acid of sequence complementary to the nucleic acid as defined above.

A nucleic acid construct comprising a polynucleotide encoding an ISUM2A polypeptide and also an expression control sequence is also referred to as “expression cassette” in the present description. In general, an expression cassette according to the invention will comprise at least one polynucleotide encoding an ISUM2A polypeptide, it being possible for the other functional elements which allow expression of the ISUM2A polypeptide to be carried by a vector into which the expression cassette can be inserted.

An expression cassette according to the invention may further comprise, without limitation, an expression control sequence and other functional elements. Nonlimiting examples of such other functional elements include leader sequences, terminator sequences, transcription initiation sequences, and stop sequences.

According to another embodiment of a nucleic acid construct of the invention, use is made of promoters known to direct the expression of the fused nucleic acid sequence in a constitutive or tissue-specific (grain) or developmental stage-specific manner. Nonlimiting examples of such expression control sequences include:
(a) constitutive promoters:
   the 35S promoter of the cauliflower mosaic virus, or the 19S promoter, or advantageously the double 35S constitutive promoter (pd35S), described in the article by Kay et al., 1987;
   the rice actin promoter followed by the rice actin intron (pAR-IAR) contained in the plasmid pAct I-F4 described by Mc Elroy et al., 1991;
   the EF-1α constitutive promoter of the gene encoding plant elongation factor described in PCT application No. WO 90/02172 or else in the article by Axelos et al. (1989);
   the chimeric super-promoter PSP (Ni et al., 1995) consisting of the fusion of three copies of the transcriptional activity element of the promoter of the octopine synthase gene from Agrobacterium tumefaciens and of the transcription-activating element of the promoter of the mannopine synthase gene from Agrobacterium tumefaciens; and
   the sunflower ubiquitin promoter (Binet et al., 1991);
   the maize ubiquitin 1 promoter (Christensen et al., 1996);
   the maize ubiquitin 1 promoter (Christensen et al., 1996).
(b) specific promoters:
   grain-specific promoters (R. Datla et al., 1997), in particular the napin promoter (EP 255 378), the phaseolin promoter (Riggs et al., 1989), the glutenin promoter, the helianthinin promoter (WO 92/17580), the albumin promoter (WO 98/45460), the oleosin promoter (WO 98/45461), the ATS1 promoter or the ATS3 promoter (WO 99/20775);
   Esr promoters as described in application PCT/FR00/02596, which allow both specific expression at the interface between the embryo and the albumen and early expression during development of the albumen;
   the promoter of the Vp1 gene described by Mc Carty et al. (1989).

The regulatory nucleic acids above can be used to overexpress the polynucleotide encoding the ISUM2A polypeptide or else a fragment of the ISUM2A polypeptide, in particular when it is desired to obtain plants having grains with large embryos rich in oil.

Thus, the invention also relates to a nucleic acid encoding an ISUM2A polypeptide or encoding a fragment of an ISUM2A polypeptide as defined above, and comprising an expression control sequence which regulates the transcription and/or the translation of the coding sequence, in which the expression control sequence allows a high level of transcription of the corresponding mRNA and/or a high level of translation of the corresponding polypeptide in the host organism, including host cell or plant, in which it is expressed.

The invention also relates to the use of a nucleic acid as defined above, of a recombinant vector comprising this nucleic acid or else of a host cell transfected or transformed with this nucleic acid, for obtaining a transformed plant capable of producing grains rich in oil.

In a preferred embodiment, controlled expression of the polynucleotide encoding the ISUM2A polypeptide is sought, most particularly when the production of grains with altered germ development is desired.

As indicated above, a deficiency in the expression of the isum2A gene causes the production of germ-free grains, these grains being infertile and not allowing multiplication of the plants mutated in the isum2A gene.

In addition, expression of the isum2A gene appears to be necessary for normal growth of the plant before pollination.

Pollination is the moment at which the mature pollen comes into contact with a receptive bristle.

It results therefrom that obtaining production of grains with altered germ development by a plant involves:
(a) normal expression of at least one functional copy of a nucleic acid encoding an ISUM2A polypeptide from the grain germination stage until pollination of the plant; and
(b) an absence of transcription or of translation of this nucleic acid once pollination of the plant has occurred, in order to produce grains altered in germ development.

The absence of transcription or of translation of the nucleic acid encoding an ISUM2A polypeptide from the stage of pollination may alter or block the development of the grain embryo in order to achieve the desired aim. However, when reproduction and multiplication of the plants of interest is sought, and when the production of normal and fertile grains is desired, normal expression of the ISUM2A polypeptide will be pursued throughout the development of the plant, including after pollination. Thus, to obtain starch-rich grains with altered germ development, it is particularly advantageous to use nucleic acid constructs in which the polynucleotide encoding an ISUM2A polypeptide is placed under the control of an expression control sequence, the activity of which can be controlled over time.

The invention therefore also relates to a nucleic acid comprising a polynucleotide encoding an ISUM2A polypeptide, chosen from the sequences having at least 95% amino acid identity with the sequences SEQ ID NO:5 and SEQ ID NO:6, and which also comprises an expression control sequence sensitive to the direct or indirect action of an inducing signal, also referred to as an inducible expression control sequence.

According to a first aspect, the expression control sequence is a transcription- or translation-“repressing” sequence. According to the invention, the expression “repressor” expression control sequence is intended to mean a regulatory sequence, the constitutive activity of which can be blocked by an external signal. Such an external signal may be the absence of binding of a transcription factor recognized by the repressor expression control sequence. The absence of binding of the transcription factor may be induced under the effect of the repressor inducer signal to which the repressor expression control sequence is sensitive.

According to this first particular embodiment, the expression of the sequence encoding an ISUM2A polypeptide is constitutive in the chosen cellular host, in the absence of the repressor inducer signal to which the repressor expression control sequence is directly or indirectly sensitive. Bringing the cellular host into contact with the repressor inducer signal has the effect, by virtue of a direct or indirect action on the repressor expression control sequence, of inhibiting and/or blocking the expression of the polynucleotide encoding the ISUM2A polypeptide. In order to produce the DNA constructs according to the invention comprising a repressor expression control sequence, those skilled in the art will make use of their technical general knowledge in the field of gene expression in plants.

According to a second embodiment of a nucleic acid construct of the invention, the expression control sequence is a transcription- or translation-activating sequence. Entirely preferably, the transcription- or translation-activating expression control sequence is directly or indirectly sensitive to the action of an activator inducer signal. This is then an “inducible activator” expression control sequence for the purpose of the invention.

According to the invention, an expression control sequence of the “inducible activator” type is a regulatory sequence which is only activated in the presence of an external signal. Such an external signal may be the binding of a transcription factor, it being possible for the binding of a transcription factor to be induced under the effect of the activator inducer signal to which the expression control sequence is directly or indirectly sensitive.

When such a nucleic acid construct is used in a cellular host, expression of the polynucleotide encoding an ISUM2A polypeptide according to the invention may be induced by bringing the transformed cellular host into contact with the activator inducer signal to which the activator expression control sequence is directly or indirectly sensitive.

When an absence of expression of the polynucleotide encoding an ISUM2A polypeptide in this transformed cellular host is sought, it is then sufficient to eliminate or suppress the presence of the activator inducer signal to which the transcription- or translation-regulating polynucleotide is sensitive.

Those skilled in the art will make use of their technical general knowledge in the field of expression control sequences, in particular those which are active in plants, to define the constructs corresponding to the definition of the second embodiment above.

In the interests of complete clarity in the explanation of the characteristics of the nucleotide constructs which are preferably used to obtain a production of germ-free grains, several nonlimiting inducible and controllable systems for the expression of an ISUM2A polypeptide in plants are defined below. The general characteristics of suitable expression systems are first of all summarized in Table 3 below, and their functional aspects are then given in detail.

TABLE 3
Examples of inducible expression systems for ISUM2A.

| | Genetic | | | | |
|---|---|---|---|---|---|
| | background | | | | |
| | of the | | | | |
| | cellular host | | | | |
| | or of the | Induction system | | Expression system | |
| | plant | Promoter | Gene | Promoter | Gene |
| I | isum2A⁻/ | Constitutive | Activator | Under the | Isum2A |
| | isum2A^{-a-} | | regulatable | control of | |
| | | | by inducer | the activator | |
| II | isum2A⁻/ | Constitutive | Repressor | Under the | Isum2A |
| | isum2A⁻ | or embryo- | regulatable | control of | |
| | | specific | by inducer | the repressor | |
| III | Isum2A⁺/ | Constitutive | Activator | Under the | Isum2A |
| | Isum2A^{+a} | or embryo- | regulatable | control of | antisense |
| | | specific | by inducer | the activator | poly- |
| | | | | | nucleotide |

^{a}isum 2A⁻/isum2A⁻: homozygous for a nonfunctional copy of the isum2A gene, for example a mutation.
^{b}Isum 2A⁺/Isum2A⁺: homozygous carrying two functional copies of the isum2A gene.

Description of the Inducible Expression System I from Table 3

The inducible expression system I from Table 3 constitutes an illustration of a nucleic acid construct in which the expression control sequence is an “inducible” transcription- and/or translation-activating” sequence according to the second embodiment defined above.

The expression system I comprises:
(i) a first expression cassette comprising a nucleic acid encoding an ISUM2A polypeptide placed under the control of a promoter, the activity of which is induced only in the presence of an activator compound bound to an inducer compound (in general, nonlimiting embodiments, this activator compound is a transcription factor);
(ii) a second expression cassette comprising a nucleic acid encoding the activator compound, for example the transcription factor above, placed under the control of a promoter which allows constitutive expression of the activator compound.

According to system I, the transcription or translation of the sequence encoding an ISUM2A polypeptide is induced only when the activator compound is complexed with the inducer compound. The complex between the activator compound and the inducer compound binds to the promoter controlling the expression of the nucleic acid encoding an ISUM2A polypeptide, and activates the expression of the latter.

An example of the system I is illustrated in Example 3, in which the system comprises:
(i) a nucleic acid encoding an ISUM2A polypeptide placed under the control of a promoter containing the Upstream Activation Sequence (UAS) sequence, which is recognized by the GVG activator compound. The GVG activator compound is a protein from fusion between the DNA-binding domain of the GAL4 protein, the VP16 gene activing domain and the rat glucocorticoid receptor (GR);
(ii) a nucleic acid encoding the GVG fusion protein defined above, which is placed under the control of a constitutive promoter.

In Example 3, the GVG fusion protein is expressed constitutively in the plant, but does not activate the promoter controlling the expression of the ISUM2A polypeptide in the absence of glucocorticoid.

On the other hand, when the plant is brought into contact with the inducer compound represented by a glucocorticoid, the glucocorticoid binds to the GVG fusion protein and the complex thus formed between the activator inducer compound (glucocorticoid) and the activator compound (the GVG fusion protein) will bind to the UAS sequence of the promoter controlling the expression of an ISUM2A polypeptide. Such binding activates the promoter containing the UAS sequence and induces the synthesis of the ISUM2A polypeptide.

In the inducible expression system above, the glucocorticoid constitutes the inducer compound. The activator inducer signal is the binding of the activator compound/inducer compound (GVG fusion polypeptide/glucocorticoid) complex to the activator expression control sequence (promoter containing the UAS sequence).

The expression system I will preferably be used in a plant cellular host or in a plant in which the two copies of the isum2A gene are nonfunctional, for example in which the two copies of the isum2A gene are mutated.

Description of the Inducible Expression System II from Table 3

The inducible expression system II is an illustration of the embodiment of a nucleic acid construct according to the invention, according to the first embodiment set out above, in which the polynucleotide encoding an ISUM2A polypeptide is placed under the control of a “repressor” expression control sequence. In the system II according to Table 3, the expression system contains two expression cassettes, respectively:
(i) a first expression cassette comprising a nucleic acid encoding an ISUM2A polypeptide, placed under the control of a regulatory region, the activity of which is constitutive because it is induced in the presence of an activator compound produced constitutively;
(ii) a second expression cassette comprising a nucleic acid encoding the activator compound which is active on the regulatory region of the expression cassette (i) above, said nucleic acid encoding the activator compound being placed under the control of a constitutive promoter, preferably a strong constitutive promoter, or else a promoter which is active specifically in the cells of the embryo of the grain.

In the system II, the activator compound loses its function of activation of the regulatory region controlling the expression of the nucleic acid encoding an ISUM2A polypeptide, when this activator compound is brought into contact with a specific ligand, which is here referred to as repressor inducer compound. Thus, in the absence of the specific ligand constituting the repressor inducer compound, the activator compound is expressed constitutively and activates the expression of an ISUM2A polypeptide.

On the other hand, when the activator compound is brought into contact with the repressor inducer compound with which it binds, the activator compound is deactivated and no longer binds to the regulatory region controlling the expression of the nucleic acid encoding an ISUM2A polypeptide. In this situation, the ISUM2A polypeptide is no longer expressed.

Example 2 illustrates a particular nonlimiting embodiment of a system II as defined above. The system II presented in Example 2 comprises, respectively:
(i) a first expression cassette comprising a nucleic acid encoding the activator compound tTA (tetracycline-controlled trans-activator protein), placed under the control of a strong constitutive promoter, the promoter of the rice actin 1 gene; and
(ii) a second expression cassette comprising a nucleic acid encoding an ISUM2A polypeptide, placed under the control of a promoter containing a tetracycline response element (TRE), the promoter being activated when the tTA activator binds to the TRE.

Thus, in the absence of any repressor inducer compound, the activator compound tTA is produced constitutively and activates the promoter controlling the sequence encoding an ISUM2A polypeptide.

On the other hand, in the presence of the repressor inducer compound, which, in this case, is tetracycline or a derivative of tetracycline, the tTA activator is deactivated and no longer binds to the TRE unit of the promoter controlling the nucleic acid encoding an ISUM2A polypeptide. In this situation, in the presence of tetracycline, the ISUM2A polypeptide is no longer expressed.

The repressor inducer signal is the absence of binding of the activator compound/repressor inducer compound (tTA activator/tetracycline) complex to the repressor expression control sequence (promoter containing the TRE unit).

Preferably, an expression system II as defined above will be used in a plant cellular host or in a plant for which the two copies of the isum2A gene are mutated or inactivated.

Description of the Inducible Expression System III from Table 3

The expression system III, which is summarized in Table 3, is very similar, with regard to the elements regulating the expression of the expression cassettes which it contains, to the expression system I above. With the expression system III, it is possible to control the expression of an antisense polynucleotide capable of inhibiting the production of an ISUM2A polypeptide in a plant comprising at least one functional copy of the isum2A gene, preferably in a plant comprising the two functional copies of the isum2A gene.

The expression system III comprises two expression cassettes, respectively:
(i) an expression cassette containing a nucleic acid encoding an antisense polynucleotide, or RNAi, which inhibits the translation of the ISUM2A polypeptide, this nucleic acid being placed under the control of a promoter which is activated by a given activator compound, when this activator compound is bound to an inducer compound; and
(ii) an expression cassette comprising a nucleic acid encoding the activator compound, this nucleic acid being placed under the control of a constitutive or embryo-specific promoter, preferably a strong promoter.

According to the expression system III, in the absence of binding of the activator compound to the inducer compound, the promoter controlling the expression of the antisense polynucleotide, or RNAi, is inactive and the antisense polynucleotide is not produced.

On the other hand, in the presence of the inducer compound capable of activating the activator compound, for example by forming a complex with it, the activated activator compound binds to the promoter controlling the expression of the isum2A antisense polynucleotide, which activates the expression of the antisense polynucleotide, leading to inhibition of the translation of the isum2A gene.

By virtue of the expression system III, the synthesis of the ISUM2A polypeptide is therefore inhibited in the presence of the inducer compound which activates the activator compound. Such an expression system makes it possible to precisely control the moment at which it is desired to block the synthesis of the ISUM2A polypeptide, by bringing the deactivated activator compound into contact with the inducer compound which activates the activator compound, for example from the beginning of pollination.

An illustrative example of an expression system III may be directly derived from that described in Example 3, when the nucleic acid encoding an ISUM2A polypeptide is substituted with a nucleic acid encoding an antisense polynucleotide which inhibits the translation of the ISUM2A polypeptide or any other method of inhibition of endogenous genes using a transgene and known to those skilled in the art (cosuppression, ribozyme, double-stranded RNA, etc).

The inducible systems for the expression of an ISUM2A polypeptide comprise several expression cassettes which may be included in a single expression vector or, on the other hand, in different expression vectors.

An inducible expression system such as the systems I, II and III described above advantageously comprises at least one selection marker gene, such as, for example, a gene for resistance to the herbicide BASTA, well known to those skilled in the art. According to a first embodiment, the selection marker gene is carried by the vector comprising the expression cassette(s) constituting the inducible expression system. According to a second embodiment, the selection marker gene is carried by a vector other than a vector comprising the expression cassette(s) constituting the inducible expression system.

An inducible activator expression control sequence selected from those described below may be used to prepare a nucleic acid construct of the invention.

Preferred Inducible Expression Control Sequences According to the Invention

The regulatory sequence capable of controlling the nucleic acid encoding an ISUM2A polypeptide according to the invention may be a regulatory sequence which can be induced by a particular metabolite, such as:
a glucocorticoid-inducible regulatory sequence, as described by Aoyama et al. (1997) or as described by Mcnellys et al. (1998);
an ethanol-inducible regulatory sequence, such as that described by Salter et al. (1998) or else as described by Caddick et al. (1998);
a tetracycline-inducible regulatory sequence, such as that sold by the company CLONTECH;
a promoter sequence which can be induced by a pathogen or by a metabolite produced by a pathogen;
a PR-type gene regulatory sequence, which can be induced by salicylic acid or BTH or Aliette (Gorlach et al., 1996, Molina et al., 1998);
a regulatory sequence of the Ecdysone receptor type (Martinez et al., 1999) which can be induced by tebufenozide (product reference RH5992, sold by Rohm & Haas) for example, belonging to the dibenzoylhydrazine family.
   Other Sequences Containing Regulatory Signal

Advantageously, the nucleic acid allowing synthesis of the ISUM2A polypeptide may also contain one or more other sequences containing regulatory signals for the expression of the region encoding ISUM2A, or alternatively may be placed under the control of such regulatory sequences.

“Leader” Sequences

The other sequences containing regulatory signals encompass 5′ untranslated sequences referred to as “leader” sequences. Such sequences may increase the translation of the mRNA encoding ISUM2A. Nonlimiting examples of said sequences include:
the EMCV leader (EncephaloMyoCarditis VIRUS 5′ noncoding region) (Elroy-Stein et al., 1989);
the TEV (Tobacco Etch Virus) leader (Carrington and Freed, 1990);
the leader of the BiP gene encoding the human immunoglobulin heavy chain-binding protein (Macejack et al., 1991);
the AMV RNA 4 leader from the mRNA of the alfalfa mosaic virus protein (Jobling et al., 1987);
the tobacco mosaic virus leader (Gallie et al., 1989).
   “Terminator” Sequences

The nucleic acid allowing synthesis of the ISUM2A polypeptide, or the vector into which this nucleic acid is inserted, may also comprise “terminator” sequences.

Among the terminators which can be used in the constructs of the invention, mention may be made, by way of nonlimiting illustration, of:
the 35S polyA of the cauliflower mosaic virus (CaMV), described in the article by Franck et al. (1980);
the nos terminator corresponding to the 3′ noncoding region of the nopaline synthase gene of the Agrobacterium tumefaciens Ti plasmid (Depicker et al., 1992);
the histone gene terminator (EP 0 633 317).

According to another alternative embodiment, the expression cassette according to the invention may comprise a polynucleotide encoding an ISUM2A polypeptide fused to a gene regulatory sequence of the glucocorticoid receptor GR fragment type (Aoyma et al. 1997), said polynucleotide being placed under the control of a promoter sequence of the native ISUM2 promoter or constitutive promoter type. In the presence of the hormone, the resulting chimeric protein is no longer retained in the cytoplasm and can therefore enter into the chloroplast and integrate into the ribosome.

Recombinant Vectors of the Invention

A nucleic acid which allows the synthesis of the ISUM2A polypeptide can be inserted into a suitable vector. For the purpose of the present invention, the term “vector” will be intended to mean a circular or linear DNA or RNA molecule which is indifferently in single-stranded or double-stranded form. A recombinant vector according to the invention is preferably an expression vector, or more specifically an insertion vector, a transformation vector or an integration vector. It may in particular be a vector of bacterial or viral origin.

In all cases, the nucleic acid which allows the synthesis of the ISUM2A polypeptide is placed under the control of one or more sequences containing signals regulating its expression in the plant under consideration, whether the regulatory signals are all contained in the nucleic acid encoding ISUM2A, as is the case in the nucleic acid constructs described in the preceding section, or whether one or more of them, or even all the regulatory signals, are contained in the recipient vector into which the nucleic acid encoding ISUM2A has been inserted.

A recombinant vector according to the invention advantageously comprises suitable transcription initiation and stop sequences. In addition, the recombinant vectors according to the invention may comprise one or more origins of replication which are functional in the host cells in which their expression is desired, and, where appropriate, selection marker nucleotide sequences.

The recombinant vectors according to the invention may also include one or more expression-regulating signals as defined above in the description.

The bacterial vectors which are preferred according to the invention are, for example, the pBR322 (ATCC No. 37 017) vectors or else the vectors such as pAA223-3 (Pharmacia, Uppsala, Sweden) and pGEM1 (Promega Biotech, Madison, Wis., United States). In addition, commercially-available vectors selected form the group consisting of pQE70, pQE60, pQE9 (Quiagen), psiX174, pBluescript SA, pNH8A, pMH16A, pMH18A, pMH46A, pWLNEO, pSV2CAT, pOG44, pXTI and pSG (Stratagene) may be used.

They may also be vectors of the Baculovirus type, such as the vector pVL1392/1393 (Pharmingen) used for transfecting cells of the Sf9 line (ATCC No. CRL 1711) derived from Spodoptera frugiperda.

In some preferred embodiments of the invention, stable and preferably inducible expression of a sequence encoding an ISUM2A polypeptide in a plant may be achieved using vectors especially suitable for expression of sequences of interest in plant cells, such as those selected form the group consisting of:
vector pBIN19 (Bevan et al.), sold by the company CLONTECH (Palo Alto, Calif., USA);
vector pBI 101 (Jefferson, 1987), sold by the company CLONTECH;
vector pBI121 (Jefferson, 1987), sold by the company CLONTECH;
vector pEGFP; Yang et al. (1996), sold by the company CLONTECH;
vector pCAMBIA 1302 (Hajdukiiewicz et al., 1994);
intermediate and superbinary vectors derived from the vectors pSB12 and pSB1 described by Japan Tobacco (EP 672 752 and Ishida et al., 1996).

The invention also provides vectors pRDP5, pRDP4, pRDP2, and pRDP3 as described in Examples 2 and 3.

Transformed Host Cells According to the Invention

In order to allow the expression of a nucleic acid encoding an ISUM2A polypeptide according to the invention, placed under the control of a suitable regulatory sequence, the recombinant nucleic acids or vectors defined in the present description must be introduced into a host cell. The introduction of the polynucleotides according to the invention into a host cell may be carried out in vitro, according to techniques well known to those skilled in the art.

A subject of the invention is also a host cell transformed with a nucleic acid according to the invention or with a recombinant vector as defined above.

Such a transformed host cell is preferably of bacterial, fungal or plant origin.

Thus, bacterial cells of various strains of Escherichia coli or else of Agrobacterium tumefaciens may in particular be used.

Advantageously, the transformed host cell is a plant cell or else a plant protoplast.

Among the cells which can be transformed according to the method of the invention, mention may be made, by way of examples, of cells of large crop plants (maize, wheat, rapeseed, sunflower, pea, soybean, barley, etc.). Preferably, plants known to contain large (protein, carbohydrate and lipid) stores, in particular cereal plants or oil-yielding plants, may be chosen.

The hybrid plants obtained by crossing plants according to the invention are also part of the invention.

Preferably, it is a cell or a protoplast of a cereal plant. The cell or the protoplast preferably comes from maize, wheat, barley, sorghum, millet, rye or rice.

Entirely preferably, it is a cell from maize.

A subject of the invention is also the use of a nucleic acid comprising a polynucleotide encoding an ISUM2A polypeptide, where appropriate in the form of a nucleic acid construct as defined above, for producing a transformed plant capable of producing grains with altered germ development.

The invention also relates to the use of a recombinant vector as defined in the present description for producing a transformed plant capable of producing grains with altered germ development.

The invention also relates to the use of a cellular host transformed with a nucleic acid comprising a polynucleotide encoding an ISUM2A polypeptide, where appropriate in the form of a nucleic acid construct or expression cassette as defined above, for producing a transformed plant capable of producing seeds with altered germ development. The invention also relates to a transformed plant comprising a plurality of host cells as defined above.

Plants Transformed with a Nucleic Acid which Allows the Synthesis of the ISUM2A Polypeptide and Methods for Obtaining Them

The invention also relates to a transformed multicellular plant organism, comprising a host cell, or a plurality of host cells, transformed with a nucleic acid comprising a polynucleotide encoding the ISUM2A polypeptide as defined in the present description, or else with a recombinant vector comprising such a nucleic acid. A subject of the invention is also a transformed plant comprising, in a form artificially integrated into its genome, a nucleic acid which allows the synthesis of the ISUM2A polypeptide, as defined in the present description.

A transformed plant according to the invention may contain a plurality of copies of a nucleic acid encoding the ISUM2A polypeptide, in situations in which overexpression of the ISUM2A polypeptide is sought. Overexpression of the ISUM2A polypeptide is sought in particular when it is desired to obtain plants producing grains in which the germ is significantly larger in size than in the “wild-type” plants, and which is enriched in oil.

According to another aspect, the overexpression of the ISUM2A polypeptide can be obtained by transforming a host cell or a plant with a nucleic acid encoding the ISUM2A polypeptide and in which the polynucleotide comprising the open reading frame is placed under the control of a regulatory nucleic acid which allows a high level of transcription of the corresponding mRNA or a high level of translation of the ISUM2A polypeptide in the host cell or in the plant.

The invention therefore also relates to a transformed plant as defined above, the grains of which are rich in oil. It also relates to a transformed plant as defined above, which has improved agronomic and/or nutritional qualities. It also relates to a method for obtaining such a transformed plant. Nonlimiting examples of transformed plants of the invention include large crop plants, preferably maize, wheat, rapeseed, sunflower, pea, soybean and barley.

In some preferred embodiments of the invention, obtaining starch-rich grains with altered germ development may be achieved by using plants with grains having a high starch content or a large amount of starch are especially preferred. Nonlimiting examples of especially preferred plants include maize, wheat, barley, sorghum, millet, rye, and rice.

The hybrid plants obtained by crossing transformed plants according to the invention are also part of the invention. The invention also relates to any part of a transformed plant as defined in the present invention, such as the root, but also the aerial parts, for instance the stem, the leaf, the flower and especially the grain.

A subject of the invention is also a plant seed or grain produced by a transformed plant as defined above. Typically, such a transformed seed or such a transformed grain comprises one or more cells comprising, in their genome, one or more copies of a nucleic acid which allows the synthesis of the ISUM2A polypeptide, where appropriate in a controlled and inducible manner.

When overexpression of the ISUM2A polypeptide has been obtained in the plant, the plant seeds or grains are enriched in oil. A subject of the invention is therefore seeds and grains enriched in oil, prepared or obtained from a transformed plant overexpressing the ISUM2A polypeptide. The grains rich in oil may be used for preparing seeds or seed meals enriched in oil, which can be used in agriculture and in the agrofoods industry.

According to a preferred embodiment of a transformed plant according to the invention, controlled expression of the ISUM2A polypeptide is sought. This implies that the only functional copy(ies) of a polynucleotide encoding the ISUM2A polypeptide in the plant is the artificially-introduced copy(ies), while the sequences of the isum2A gene that are found naturally in the wild-type plant carry at least one mutation causing a deficiency in expression of the isum2A gene.

Such plants mutated in the isum2A gene are, for example, the G2422 mutant plants or the emb*-8516 mutant plants described by Heckel et al. (1999). Those skilled in the art can, by virtue of the invention, produce other plants mutated in the isum2A gene, for example by random insertion of the Mutator transposon into a population of plants of wild-type phenotype (Isum2A⁺/Isum2A⁺), and then detection in the mutants obtained of those among these mutants which no longer express the isum2A gene, for example using the nucleotide probes or primers described in the examples.

According to this preferred embodiment, the transformed plant according to the invention is characterized in that it derives from a plant in which the two copies of the isum2A gene each carry at least one mutation causing a deficiency in its expression, at the transcriptional or translational level.

According to a second preferred embodiment according to the invention, controlled inhibition of the synthesis of the ISUM2A polypeptide, for example through the expression of an antisense polynucleotide, is sought. In this case, the transformed plant comprises at least one functional copy of the isum2A gene, and preferably the two copies of the isum2A gene are functional.

The invention also relates to a method for obtaining a transformed plant capable of producing grains altered in germ development, comprising:
(a) transforming at least one plant cell;
   with a nucleic acid comprising a polynucleotide encoding an ISUM2A polypeptide, chosen from the sequences having at least 95% amino acid identity with the sequences SEQ ID NO:5 and SEQ ID NO:6, said nucleic acid also comprising an expression control sequence as defined in the present description; or
   with a recombinant vector comprising such a nucleic acid;
(b) selecting the cells transformed in step (a) which have integrated into their genome at least one copy of a nucleic acid encoding the ISUM2A polypeptide;
(c) regenerating a transformed plant from the transformed cells obtained in step (b).

According to the invention, the expression “grains altered in germ development” is intended to mean grains in which the development of the germ is “abnormal”, i.e. differs significantly from the germ of a “wild-type” plant grain.

According to a first aspect, the germ development may be altered such that the size of the germ is significantly greater than that of the germ found in the grains of “wild-type” plants not altered in isum2A gene expression. A significantly increased size of the germ can be obtained by overexpressing the ISUM2A polypeptide, for example either by introducing a plurality of copies of a nucleic acid encoding this polypeptide into a host cell or into a plant, or by placing a copy of the isum2A gene or of its cDNA under the control of an expression control sequence which allows overexpression of the ISUM2A polypeptide in the host cell or in the plant.

By overexpressing the ISUM2A polypeptide from the early stage of embryo development, large grains rich in oil may be obtained.

A subject of the invention is also a particular embodiment of the method above for obtaining a transformed plant capable of producing grains with altered germ development, in which at least one plant cell is transformed in step (a) with Agrobacterium tumefaciens containing:
a nucleic acid comprising a polynucleotide encoding an ISUM2A polypeptide, chosen from the sequences having at least 95% amino acid identity with the sequences SEQ ID NO:5 and SEQ ID NO:6 and comprising an expression control sequence as defined in the present description; or
a recombinant vector comprising such a nucleic acid.

Each of the methods for obtaining a transformed plant according to the invention may comprise the following additional steps:
d) crossing a plant selected in step c) with a heterozygous plant comprising a functional copy of the isum2A gene and an inactive copy of the isum2A gene;
e) selecting the plants derived from the cross of step d) which are homozygous and carry two inactive copies of the isum2A gene.

Preferably, the expression control sequence is sensitive to the action of an inducer signal; the expression control sequence is also referred to as inducible.

According to a first preferred embodiment, the inducible expression control sequence consists of a “repressor” expression control sequence as defined in the present description.

According to a second preferred embodiment, the inducible expression control sequence is a transcription- or translation-“activating” expression control sequence as defined in the present description.

The present invention also relates to a transformed plant as obtained by one of the methods of production defined above.

The invention also relates to a hybrid transgenic plant obtained by crossing a transformed plant as defined above.

The invention also relates to a part of a transformed plant according to the invention.

A subject of the invention is also a method for obtaining plant grains with altered germ development, comprising:
(a) cultivating, until pollination, a plant in which the two copies of the isum2A gene carry at least one mutation causing a deficiency in the production of the ISUM2A polypeptide, and into the genome of which has been artificially introduced a nucleic acid comprising:
   a polynucleotide encoding an ISUM2A polypeptide, chosen from the sequences having at least 95% amino acid identity with the sequences SEQ ID NO:5 and SEQ ID NO:6; and
   an inducible expression control sequence of the repressor type controlling the expression of the polynucleotide encoding the ISUM2A polypeptide,
   the cultivating of the plant being carried out in the absence of the repressor inducer signal to which the repressor expression control sequence is sensitive;
(b) bring the transformed plant defined in (a) into contact with the repressor inducer signal to which the repressor expression control sequence is sensitive, for a period of time ranging from pollination to the end of grain formation;
(c) recovering the mature grains, characterized in that they are altered in germ development.

The germ development can be altered such that the size of the germ is significantly decreased compared to that of the grains originating from wild-type plants, or even nonexistent, as has been shown when the plant has a nonfunctional isum2A gene or else when a nucleic acid encoding the ISUM2A polypeptide under the control of an expression control sequence which makes it possible to block the transcription or translation in a controlled manner in order to alter germ development is introduced.

A subject of the present invention is also a method for obtaining plant grains with altered germ development, comprising:
(a) cultivating, until pollination, a plant in which the two copies of the isum2A gene each carry at least one mutation causing a deficiency in the production of the ISUM2A polypeptide, and into the genome of which has been artificially introduced a nucleic acid comprising:
   a polynucleotide encoding an ISUM2A polypeptide, chosen from the sequences having at least 95% amino acid identity with the sequences SEQ ID NO:5 and SEQ ID NO:6; and
   an expression control sequence of the inducible activator type controlling the expression of the polynucleotide encoding the ISUM2A polypeptide,
   the cultivating of the plant being carried out in the presence of the activator inducer signal to which the activator expression control sequence is sensitive;
(b) continuing the cultivation of the transformed plant defined in (a) in the absence of the activator induced signal to which the activator expression control sequence is sensitive, from the period following pollination;
(c) recovering the mature grains, characterized in that they are altered in germ development.

The invention also relates to a seed with altered germ development as obtained according to one of the methods defined above.

The invention also relates to a seed with altered germ development, characterized in that each of its constitutive cells comprises, in a form artificially integrated into their genome, a nucleic acid comprising:
a polynucleotide encoding an ISUM2A polypeptide, chosen from the sequences having at least 95% amino acid identity with the sequences SEQ ID NO:5 and SEQ ID NO:6; and
an expression control sequence.

According to a first embodiment, the expression control sequence consists of an inducible expression control sequence of the repressor type.

According to a second preferred embodiment, the expression control sequence consists of an expression control sequence of the inducible activator type.

Also part of the invention is any product of transformation of the seed as defined in the present description, in particular a seed or an oil.

Preferably, the product of transformation is a starch.

In order to obtain starch from a seed with altered germ development according to the invention, those skilled in the art will advantageously make use of the techniques described in the work “Handbuch der Starke” (vol. 1; Max Ullmann (ed.), Paul Varey Verlag, Berlin) or else in the article by Morrison and Karkalas (Methods in Plant Biochemistry, 1990, vol.2: 323-352, Academic Press Ltd; London).

The starch obtained from the seeds with altered germ development according to the invention may be used by the agrofoods industry, the pharmaceutical industry or the paper industry, or else in the microbiological field, where it can be used as a nutritive substrate.

The transformation of plant cells can be carried out by the techniques known to those skilled in the art. Nonlimiting examples include direct microinjection into plant embryoids (Neuhaus et al., 1987), infiltration under vacuum (Bechtold et al., 1993) or electroporation (Chupeau et al., 1989) or else direct precipitation by means of PEG (Schocher et al., 1986) or bombardment, using a particle gun, of particles coated with the plasmid DNA of interest (Fromm M. et al. 1990).

It is also possible to infect the plant with a bacterial strain, in particular of Agrobacterium. According to one embodiment of the method of the invention, the plant cells are transformed with a vector according to the invention, said cellular host being capable of infecting said plant cells by allowing integration into the genome thereof of the nucleotide sequences of interest initially contained in the DNA of the abovementioned vector. Advantageously, the abovementioned cellular host used is Agrobacterium tumefaciens, in particular according to the method described in the article by An et al., (1986), or else Agrobacterium rhizogenes, in particular according to the method described in the article by Guerche et al., (1987) or else in PCT application No. WO 00/22148.

For example, the transformation of the plant cells can be carried out by transferring the T region of the tumor-inducing extrachromosomal circular plasmid Ti from Agrobacterium tumefaciens, using a binary system (Watson et al. 1994). To do this, two vectors are constructed. In one of these vectors, the T region has been removed by deletion, with the exception of the right and left borders, and between them the gene of interest and also a marker gene are inserted, so as to allow selection in the plant cells. The other partner of the binary system is an auxiliary plasmid Ti, which modified plasmid no longer has a T region but still contains the vir virulence genes required for transformation of the plant cell.

According to a preferred embodiment, use may be made of the method described by Ishida et al. (1996) for the transformation of monocotyledons. According to another protocol, the transformation is carried out according to the method described by Finer et al. (1992) using a particle gun with tungsten or gold particles.

Those skilled in the art are capable of implementing many methods of the state of the art in order to obtain plants transformed with a nucleic acid which allows synthesis of the ISUM2A polypeptide.

Those skilled in the art may advantageously refer to the technique described by Bechtold et al. (1993) in order to transform a plant using the bacterium Agrobacterium tumefaciens. Techniques using other types of vectors may also be used, such as the techniques used by Bouchez et al. (1993) or else by Horsch et al. (1994). By way of illustration, a transgenic plant according to the invention may be obtained by biolistic techniques such as those described by Finer et al. (1992) or else those described by Vain et al. (1993).

Other preferred techniques for transforming a plant in accordance with the invention with Agrobacterium tumefaciens are those described by Ishida et al. (1996) or else in the PCT application published under the No. WO 95/06722 in the name of Japan Tobacco.

Polypeptides Encoded by the ISUM2A Gene

As already described above, the isum2A gene encodes a polypeptide 143 amino acids in length, for which it has been possible to observe at least two variant polypeptides.

The first variant polypeptide encoded by the isum2A gene has the amino acid sequence SEQ ID NO:5 and is encoded by the isum2A gene present in the genome of the maize plant denoted HD5×HD7.

The second variant ISUM2A polypeptide is encoded by the isum2A gene present in the genome of the maize plant denoted A188.

The ISUM2A polypeptides of sequences SEQ ID NO:5 and SEQ ID NO:6 differ only by virtue of the substitution of an amino acid residue, the polypeptide of sequence SEQ ID NO:5 having a glycine residue at position 89 and the polypeptide of sequence SEQ ID NO:6 having an asparagine amino acid residue at this position.

The expression of one or other of the ISUM2A polypeptide variants leads, in all cases, to the expression of a wild-type phenotype in the plant, said plant producing mature, fertile grains comprising a completely developed embryo.

A homology search in the PROSITE database has made it possible to show structural similarities between the ISUM2A polypeptides according to the invention and the chloroplast L35 proteins.

The ISUM2A polypeptide of amino acid sequence SEQ ID NO:5 has a calculated molecular weight of 15112 daltons, a calculated isoelectric point of 11.75 and a charge at pH 7 of 28.19.

The ISUM2A polypeptide of sequence SEQ ID NO:5 has 40 charged amino acid residues (R, K, H, Y, C, D, E), 3 acidic amino acid residues (D, E), 31 basic amino acid residues (K, R), 31 polar amino acid residues (N, C, Q, S, T, Y) and 55 hydrophobic amino acid residues (A, I, L, F, W,V).

Given the presence of a single substitution of an amino acid residue, with respect to the sequence SEQ ID NO:5, the ISUM2A polypeptide of sequence SEQ ID NO:6 has characteristics very similar to those described above for the ISUM2A polypeptide of sequence SEQ ID NO:5.

A subject of the invention is therefore also the polypeptide comprising the amino acid seqence SEQ ID NO:5 or SEQ ID NO:6 and also a polypeptide having at least 95% amino acid identity with the sequence SEQ ID NO:5 or SEQ ID NO:6, or a fragment or a variant thereof.

A fragment of an ISUM2A polypeptide according to the invention comprises at least 10, 15, 20, 25, 30, 40, 50, 60, 70, 80, 90, 100, 120, 130, 135 or 140 consecutive amino acids of a polypeptide of sequence SEQ ID NO:5 or SEQ ID NO:6.

Also part of the invention is a polypeptide comprising 10, 15, 20, 25, 30, 40, 50, 60, 70, 80, 90, 100, 120, 130, 135 or 140 consecutive amino acids of a polypeptide of sequence SEQ ID NO:5 or SEQ ID NO:6.

The invention also relates to a polypeptide comprising an amino acid sequence having at least 95% amino acid identity with the sequence of an ISUM2A polypeptide of sequence SEQ ID NO:5 or SEQ ID NO:6.

Advantageously, also part of the invention is a polypeptide having at least 96%, 97%, 98%, 99%, 99.1%, 99.2%, 99.3%, 99.4%, 99.5%, 99.6%, 99.7%, 99.8% or 99.9% amino acid identity with the sequence of a polypeptide of sequence SEQ ID NO:5 or SEQ ID NO:6, or a peptide fragment thereof.

In general, the polypeptides according to the present invention are in an isolated or purified form.

Another subject of the invention consists of a polypeptide comprising amino acid modifications of 1, 2, 3, 4 or 5 substitutions, additions or deletions of an amino acid compared to the amino acid sequence of a polypeptide of sequence SEQ ID NO:5 or SEQ ID NO:6 or else of a fragment or of a variant thereof.

A polypeptide according to the invention may be obtained by genetic recombination according to techniques well known to those skilled in the art, for example techniques described in AUSUBEL et al. (1989).

A polypeptide according to the invention may also be prepared by conventional techniques of chemical synthesis, equally in homogeneous solution or in solid phase.

By way of illustration, a polypeptide according to the invention may be prepared by the homogeneous solution technique described by HOUBEN WEIL (1974) or else by the solid-phase synthesis technique described by MERRIFIELD (1965a; 1965b).

Preferably, the polypeptides which are variants of a polypeptide according to the invention conserve their ability to be recognized by antibodies directed against the polypeptides of sequence SEQ ID NO:5 or 6.

A polypeptide encoded by the Isum2A gene according to the invention, such as a polypeptide of amino acid sequence SEQ ID NO:5 or 6, or else a variant or a peptide fragment thereof, is useful in particular for preparing antibodies intended for the detecting the presence and/or the expression of a polypeptide of sequence SEQ ID NO:5 or 6 or of a peptide fragment thereof, in a sample.

Besides the detection of the presence of a polypeptide encoded by the Isum2A gene or else of a peptide fragment of such a polypeptide, in a sample, antibodies directed against these polypeptides are used for quantifying the synthesis of a polypeptide of sequence SEQ ID NO:5 or 6, for example in cells of a plant, and thus determine the development of the embryo.

For the purpose of the present invention, the term “antibodies” will be intended to mean in particular polyclonal or monoclonal antibodies or fragments (for example F(ab)′₂, F(ab) fragments) or else any polypeptide comprising a domain of the initial antibody, which recognizes the target polypeptide or the target polypeptide fragment according to the invention.

Monoclonal antibodies may be prepared from hybridomas according to the technique described by KÖHLER and MILSTEIN (1975).

The present invention also relates to antibodies directed against a polypeptide as described above or a fragment or a variant thereof, as produced in the trioma technique or else the hybridoma technique described by KOZBOR et al. (1983).

The invention also relates to single-chain Fv (ScFv) antibody fragments as described in U.S. Pat. No. 4,946,778 or else by MARTINEAU et al. (1998).

The antibodies according to the invention also comprise antibody fragments obtained by means of phage libraries as described by RIDDER et al. (1995) or else humanized antibodies as described by REIWANN et al. (1997) and LEGER et al. (1997). The preparations of antibodies according to the invention are useful in immunodetection assays intended to identify the presence and/or or the amount of a polypeptide of sequence SEQ ID NO:5 or 6, or of a peptide fragment thereof, present in a sample.

An antibody according to the invention may also comprise an isotopic or nonisotopic detectable label, for example a fluorescent label, or else may be coupled to a molecule such as biotin, according to techniques well known to those skilled in the art.

Thus, a subject of the invention is also a method for detecting the presence of a polypeptide in accordance with the invention in a sample, said method comprising the steps of:
(a) bringing the test sample into contact with an antibody as described above;
(b) detecting the antigen/antibody complex formed.

The invention also relates to a diagnostic pack or kit for detecting the presence of a polypeptide in accordance with the invention in a sample, said pack comprising:
(a) an antibody as defined above;
(b) where appropriate, one or more reagents required for the detection of the antigen/antibody complex formed.

Another subject of the invention consists of the use of a nucleic acid or of an allelic variant of a nucleic acid as defined above, in selection programs for obtaining plants with modified embryo size and/or development influencing the starch and/or oil content.

The invention also relates to a method for selecting plants with modified embryo size and/or development, comprising the steps of:
(a) genotyping plants (individuals) using nucleotide probes or primers obtained from the nucleic acids as defined above or variants of these nucleic acids;
(b) selecting, from these plants (individuals), those which comprise a high frequency of favorable alleles associated with the size and/or development of the embryo.

### EXAMPLES

The present invention is also illustrated, without however being limited, by the following examples.

### Example 1

### Cloning and the Genomic Sequence of the Isum2A Gene

A. Materials and Methods

A.1. Plant Materials

Plants of the A188 line (Gerdes and Tracy, 1993) were used for the “genomic walk” and the isolation of RNA. As regards the DNA fragments contained in the genomic library which was screened, they originate from the hybrid line HD5×HD7 (Barloy et al., 1989). The mutant emb*-8516 and the recurrent parent Rscm2 were described in Heckel et al. (1999).

A.2. Cultivating of Plants

The plants were cultivated either in a climatic chamber, with a light period of 16 h (100 Wm-2), a relative humidity of 80% and a 24° C./19° C. (day/night) alternation, or in a greenhouse under the same conditions but without humidity control, or in an open field in Lyons. All the plants were pollinated by hand.

A.3. Isolation of Plant Genomic DNA

A young maize leaf 10 cm long was removed, placed in a 1.5 ml Eppendorf tube and ground with a suitable pestle, in liquid nitrogen. 500 μl of extraction buffer (100 mM Tris-HCl, pH 8, 50 mM EDTA pH 8, 100 mM NaCl, 1% SDS) were added and the tube was incubated for 5 min at 55° C. After two extractions with phenol/chloroform, the supernatant was precipitated using 50 μl of sodium acetate and 350 μl of isopropanol (cold) for 10 min at ambient temperature. The DNA pellet was rinsed with 1 ml of 70% ethanol, dried and taken up in 50 μl of TE10.01 (10 mM Tris-HCl, pH 8, 0.1 mM EDTA pH 8) supplemented with 20 μg/ml of RNAse A.

A.4. PCR on Genomic DNA

The genomic DNA was diluted 10-fold and 2 μl were used in a 20 μl reaction containing 1 μM of each primer, 100 μM of dNTP and 1 u of Taq polymerase in the buffer provided with the enzyme (Pharmacia). The reaction was carried out in a Perkin Elmer DNA thermal Cycler 2400 or 9700 PCR amplifier in the following way: after denaturation of the DNA for 2 min at 94° C., the samples were subjected to 30 cycles of denaturation for 30 s at 94° C./hybridization for 45 s at 60° C./elongation for 1 min at 72° C. All the primers had Tm values between 60° C. and 62° C.

A.5. Cloning

The plasmid DNA was prepared according to the alkaline lysis protocol described by Sambrook et al. (1989). The digestions of genomic DNA, of plasmid DNA or of lambda phage DNA with restriction enzymes were carried out according to the manufacturers' (Gibco, Boehringer Mannhein, Promega) recommendations. The products from digestion or from PCR amplification were separated by agarose gel electrophoresis in TAE buffer (Sambrook et al., 1989). The restriction fragments were ligated into the vector pBluescript and the PCR products were ligated into pGEM-T-easy, by means of T4-DNA ligase according to the supplier's (Promega) recommendations, using 50 ng of vector, and an insert/vector molar ratio of the order of 3/1. These constructs were introduced into the bacterial strain DH5-α by heat-shock transformation, according to the protocol of Hanahan (1983).

A.6. DNA Hybridization

Membranes for DNA hybridization were obtained either by transfer of agarose gel restriction fragments onto Hybond N+membranes (Amersham) by capillarity, in the presence of 0.4 N sodium hydroxide, or by transfer of lambda phage lysis plaques onto Hybond N membranes (Amersham). The membranes were prehybridized according to the supplier's recommendations, for 4 to 12 h at 65° C. in the presence of 5×SSPE (20×SSPE contains 3.6 M NaCl, 0.2 M sodium phosphate, EDTA, pH 7.7; 0.02 M). They were then hybridized for 16 h under the same conditions. The probes used for the hybridization were radioactively labeled with 50 μCi of α³²P-dCTP, using the Ready-To-GO™ DNA labelling beads kit (Amersham). After hybridization, the membranes were subjected to four rinses, at 65° C. in solutions containing 0.1% of SDS and SSC concentrations of 2×, 1×, 0.5× and 0.1×(20×SSC contains M NaCl₃, 0.3 M Na₃-citrate), and were exposed to films for autoradiography (X-OMAT, Kodak).

A. 7. Genomic DNA Library Screening

A genomic DNA library contained in the EMBL3 SP6/T7 lambda phage (Stratagene) was plated out at a density of 1.5×10⁵ lysis plaques per dish, on 10 dishes, and then transferred onto Hybond N membrane according to the supplier's (Amersham) recommendations. After hybridization and autoradiography, the lysis plaques giving a signal were removed from the dishes and plated out at a lower density, and thus twice in a row in order to obtain a single clone per dish, in the form of isolated plates. The DNAs were then prepared according to Sambrook et al. (1989).

A.8. AIMS (“Amplification of Insertion Mutagenized Sites”)

The amplification of insertion mutagenized sites was obtained according to Frey et al. (1998). The Tru9A enzyme (Promega) was used for the digestion in place of Msel. The sequences of the “Mu-specific” and “Mu-nested” primers were degenerate at additional positions. The primers used, Mu15 (SEQ ID NO:14) and Mu16 (SEQ ID NO:8), are listed in Table 4.

A.9. “Genomic Walk”

The protocol of Devic et al. (1997) was applied without modification using the Dral, EcoRV, PvuII, ScaI and SspI enzymes. The PCR products were cloned into the vector pGEM®-T-Easy (Promega).

A.10. RT-PCR

Total RNA was extracted from 50 mg of ground tissue in liquid nitrogen with the TRIZOL™ reagent according to the supplier's (Gibco) protocol. The RNA, resuspended in 50 μl of Versol water (Aguettant), was denatured at 65° C. for 5 min and treated with DNase I according to the supplier's (Promega) indications. It was then purified by two volume for volume extractions with phenol/chloroform, pH 4.5, and precipitation with ethanol. 6 μg of purified RNA were then “reverse” transcribed with MuLV reverse transcriptase (Gibco) in a total volume of 20 μl, as recommended by the supplier, with a polyT primer at a final concentration of 2.5 μM. After inactivation of the reverse transcriptase by heating at 95° C. for 5 min, 2 μl of the reverse transcription reaction were amplified with Taq polymerase as described above.

A.11. Sequencing

The sequencing was carried out by the company Genome Express in Grenoble (France). The Sequencher 3.1.1 (Gene Codes Corporation) and DNAstar (Laser Gene) software made it possible to analyze and assemble the sequences obtained.

B. Results

B.1. Cloning of the Sequences Flanking the Instertion of Mutator in emb*-8516

a) Cloning of a Flanking Sequence

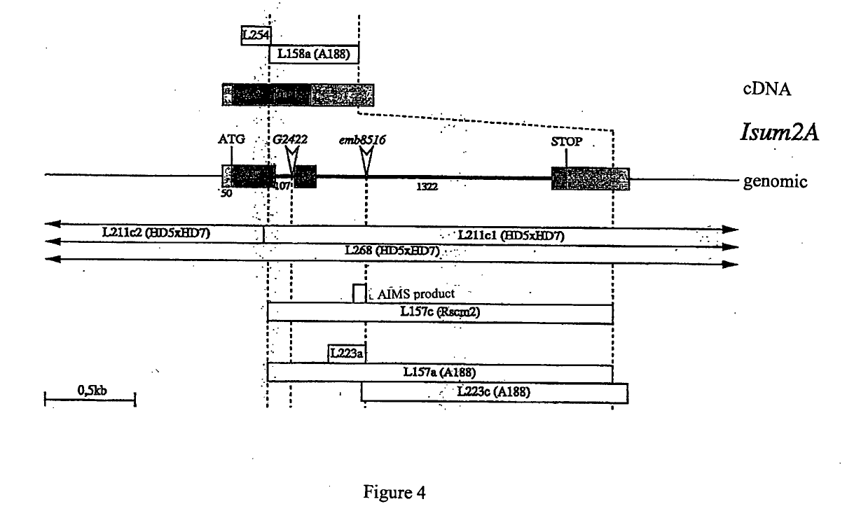

As described in Heckel et al. (1999), a 107 bp AIMS product was obtained with the primers Mu16 (SEQ ID NO:8) and adapMseI (SEQ ID NO:9). This “AIMS product” (see FIG. 4) contains 70 pb of the flanking sequence between the two primers, which does not show any significant homology with the sequences referenced in the databases.

b) Extension of the Flanking Sequence

In order to extend this flanking sequence, the “genomic walk” (Devic et al., 1997) technique was used on the genomic DNA of the A188 genotype. With the nested primers GW4 (SEQ ID NO:12) and GW4b (SEQ ID NO:13) and cleavage with SspI, a 244 bp product was obtained (clone L223a, FIG. 4), which contained an additional 153 bp of the flanking sequence.

To characterize the sequence on the other side of the insertion, the “genomic walk” technique was used with the “nested” primers GW3 (SEQ ID NO:10) and GW3b (SEQ ID NO:11) with SspI cleavage. The 1532 bp product (clone L223c, FIG. 4) contained 1463 bp of additioinal flanking sequence.

c) Homology of the Flanking Sequence in the Databases

Contrary to the “AIMS product” sequence and the sequence of the L223a clone, the sequence of the L223c clone showed homology in the databases with a maize EST. Two sequences of the same cDNA are found in Genebank: a sequence from the 5′ end in accession AI001298, and a sequence from the 3′ end in accession A1374506. This EST bears the name Isum2, without any explanation of this acronym. The homology was strong but restricted to a small portion of L223c.

A PCR reaction was carried out on genomic DNA with the primers Isum2b (SEQ ID NO:18) and Isum2e (SEQ ID NO:20), which are located on either side of the presumed intron. The amplification product was cloned and sequenced. The sequence of this clone L157a (FIG. 4) showed that the sequence present in the AIMS product, in L223a and in a portion of L223c corresponded to an intron of Isum2.

B.2. Isolation of a Second Allele

(a) Screening of a Collection of Insertion Mutants by Reverse Genetics (“Gene Machine”)

A population of 25 000 plants highly mutagenized with the mutator transposon was screened by PCR for an insertion in Isum2. The plants were planted in 10 blocks of 50×50 plants. Pools of DNAs from the 500 rows and 500 columns were analyzed by PCR for the presence of an amplification with the primer OMuA (SEQ ID NO:24 in mutator) in combination with a primer in an exon of Isum2. The four primers Isum2b (SEQ ID NO:18), Isum2c (SEQ ID NO:19), Isum2k (SEQ ID NO:21) and 8516g (SEQ ID NO:23) specific for Isum2 were tested.

Positive results were obtained with the primers Isum2b and Isum2k. The plants in question were identified and the amplification was confirmed on individual plant DNA. Their descendants were then analyzed in order to distinguish somatic insertions (absent in the gametes) from germinative insertions (present in the gametes). Of all the insertions, only one was found in the subsequent generation. Sequencing of the PCR amplification products showed that it was an insertion in intron 1 of Isum2A, 3 bp upstream of exon 2, found in a plant denoted G2422.

The grains of this plant G2422 were analyzed for the presence of the “germ-free grains” phenotype. The presence of many “parasitic” mutations in this highly mutagenized material made it difficult to evaluate some of the grains. Grains with a “germ-free grain” phenotype were detected.

(b) Lack of Complementation Between the Two Mutated Alleles of isum2A.

To prove that the “germ-free grain” phenotype of the emb*-8516 mutant described by Heckel et al. (1999) was also caused by the insertion of mutator into the Isum2A gene, a complementation between the emb*-8516 mutant and descendants of the plant G2422 with an insertion of mutator into the same Isum2A gene was undertaken.

Sixteen heterozygous +/emb*-8516 plants were pollinated by 6 heterozygous +/emb*-2422 plants. In no case was the emb*-8516 mutation complemented. This shows that the G2422 plants carry a mutation in the same gene which causes the emb phenotype of the emb*-8516 mutant. Since both have an insertion of mutator into the Isum2A gene, it is established that this mutation of the isum2A gene causes the “germ-free grain” phenotype.

B.3. The Isum2A Gene

(a) Genomic Sequences of Isum2A

For the Isum2A gene, a first genomic sequence was obtained by screening a genomic library of plants of the HD5×HD7 genotype, and a second genomic sequence was obtained by PCR on plants of the A188 genotype. The clones used for the sequencing are shown in FIG. 4.

The sequence of the HD5×HD7 genotype (FIG. 1) comes from the two subclones L211c1 (9 kb XhoI fragment) and L211c2 (6 kb XhoI fragment).

The sequence of the A188 genotype (FIG. 2) originates from the clones L157a (PCR with the primers Isum2b (SEQ ID NO:18) and Isum2e (SEQ ID NO:20)), L223a (genomic walk, see above) and L223c (genomic walk, see above).

(b) cDNA Sequences

The partial cDNA sequence of the A188 genotype (SEQ ID NO:4, FIG. 3) originates from the L158a and L254 clones (FIG. 4). The first was obtained by RT-PCR on 12 JAP embryos with the primers Isum2b (SEQ ID NO:18) and Isum 2c (SEQ ID NO:19), and the second by RT-PCR on 7JAP albumens with the primers Isum2a (SEQ ID NO:17) and Isum2l (SEQ ID NO:22).

The cDNA sequence of the genotype HD5×HD7 (SEQ ID NO:3, FIG. 2) was obtained from the genomic sequence (SEQ ID NO:1) using the Isum2 EST (above) to determine the 5′ limit of the first exon and the 3′ limit of the last exon.

Table 4 below gives details of the various primers used in this example.

TABLE 4
Primers used

| | | | |
|---|---|---|---|
| | | SEQ ID | |
| Name | 5′ to 3′ sequence | NO. | Use |

| | | | |
|---|---|---|---|
| Mu16 | TCYATAATGGCAATTATCTC | 8 | AIMS product |
| adapMseI | GATGAGTCCTGAGTAAN | 9 | AIMS product |
| GW3 | GAAACTGGAAAGGCGAAATGGAGGGACG | 10 | L223c |
| GW3b | GCTCGATAGGTTTATTGTGATAACGTTGCTGC | 11 | L223c |
| GW4 | GTCCCTCCATTTCGCCTTTCCAGTTTCC | 12 | L223a, |
| GW4b | CCAGCAACGTTATCACAATAAACCTATCGAGC | 13 | L223a |
| Mu15 | GAGAAGCCAACGCCAWCGCCTCYATTTCGTC | 14 | AIMS product |
| AP1 | GGATCCTAATACGACTCACTATAGGGC | 15 | all “genomic |
| | | | walk” |
| AP2 | CTATAGGGCTCGAGCGGC | 16 | all “genomic |
| | | | walk” |
| Isum2a | CTACCCGCAGCCAGCCTCGCATTCC | 17 | L254 |
| Isum2b | GGCGGGGAAGAAGGGCTACAAGATGAAGAC | 18 | GM, L158a1, |
| | | | L157a |
| Isum2c | GCCAAGAAGAACACCAAGCGCAAGAAGAG | 19 | GM^{a} |
| Isum2e | CGATCTGCTGGCCATATCCTAAGAG | 20 | L158a1, |
| | | | L157a |
| Isum2k | CATCTTCGAGAGCCTCTTCTTGCG | 21 | GM |
| Isum2l | CTTCATCTTGTAGCCCTTCTTCCCCG | 22 | L254 |
| 8516g | GCACCCGTAACATTGTCGTAGTC | 23 | GM |
| OMuA | CTTCGTCCATAATGGCAATTATCTC | 24 | GM |

^{a}GM means “gene machine”.

### Example 2

### Construction of Vector Comprising a Polynucleotide Encoding an ISUM2A Polypeptide Placed Under the Control of an Inducible Expression Control Sequence of the Repressor

The expression system described in this example constitutes an illustration of the expression system II summarized in Table 3, for which details of the general principle of functioning are given in the description.

This expression system involves the construction of two vectors, respectively:
(i) the vector pRDP5, whcih contains the isum2A gene placed under the control of the TRE sequence recognized by the tTA activator; and
(ii) the vector pRDP4, which contains the gene of the tTA activator, which is active in the absence of tetracycline, placed under the control of the rice actin1 promoter, which is a strong and constitutive promoter in monocotyledons.
   A.1. Construction of the Vector pRDPS

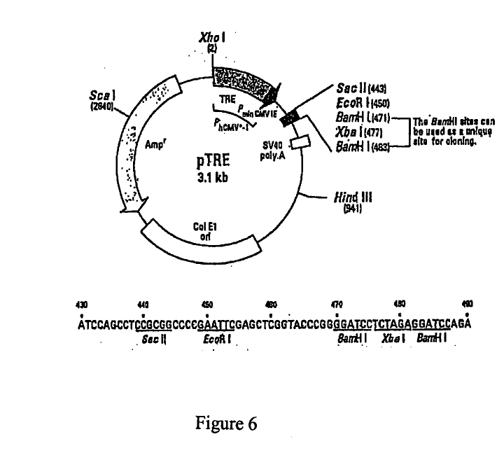

The vector pTRE, sold by the company CLONTECH Laboratories Inc., and which is also described in the GENBANK database under the accession number U89931, is used as starting vector (FIG. 6). The vector pTRE is cleaved with the BamHI restriction endonuclease, at nucleotides 471 and 483 of the vector, the DNA fragment located between the abovementioned BamHI sites then being excised.

After cleavage and excision, the sticky ends are filled in using Klenow polymerase.

The isum2A gene described in Example 1, more specifically the genomic region ranging from the ATG codon to the end of the reading frame and also comprising a terminator sequence, is then amplified by PCR.

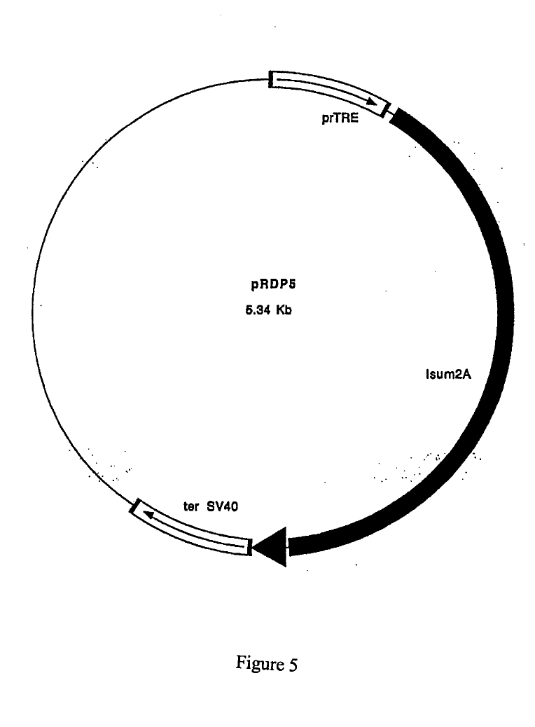

The isum2A gene amplification product is then ligated into the open pTRE vector in order to construct the vector pRDP5 illustrated in FIG. 5.

In the vector pRDP5, the isum2A gene is placed under the control of the prTRE promoter, which is recognized by the tTA activator.

A.2. Construction of the Vector pRDP4

The vector pRDP4 contains the gene of the tTA activator under the control of the rice actin1 promoter.

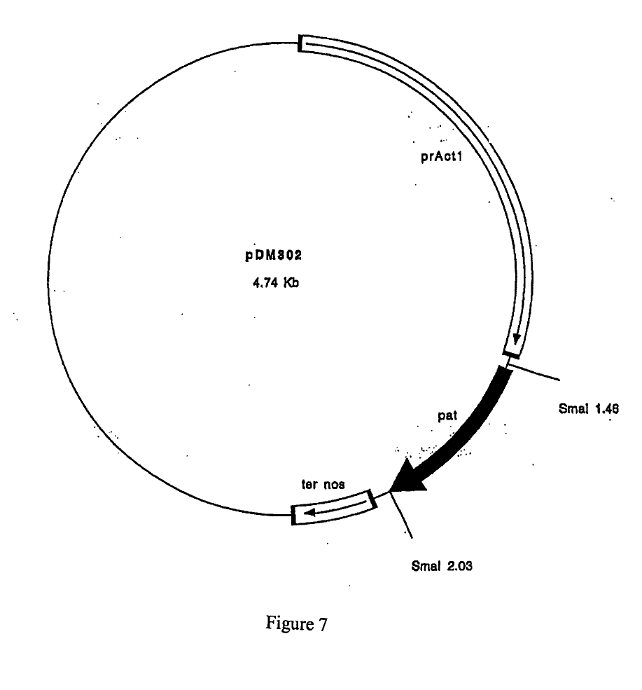

The starting vector is the plasmid pDM302, which was described by CAO et al. (1992) and which is illustrated in FIG. 7.

The plasmid pDM302 is digested with the SmaI restriction endonuclease.

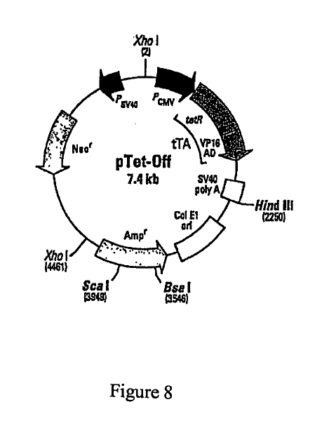

The vector pTet-Off sold by the company CLONTECH Laboratories Inc. (catalog reference No. K 1620-A, from the year 2000), which is illustrated in FIG. 8, is then used to amplify the sequence of the tTA gene by PCR.

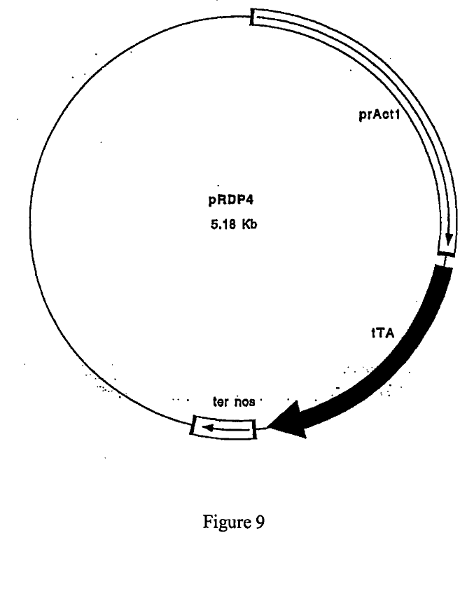

The tTA gene amplification product is then ligated into the open SmaI site of the plasmid pDM302 in order to construct the plasmid pRDP4 illustrated in FIG. 9.

The plasmid pRDP4 comprises the tTA gene under the control of the rice actin1 promoter.

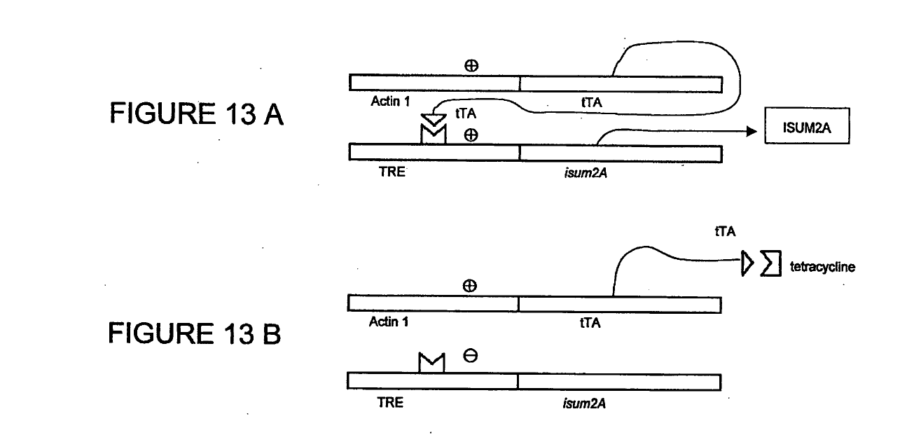

A diagram for the functioning of the inducible expression system described in the present example is represented in FIG. 13, even in the absence of tetracycline (FIG. 13A), or in the presence of tetracycline (FIG. 13B).

A summary of the characteristics of the various vectors used or prepared according to Example 2 is given in Tables 5 to 8 below.

TABLE 5
Positions of the characteristic elements of the
plasmid pDM302 (unit: kilobase)

| | | |
|---|---|---|
| vector pSP72 | 0.00 | 0.04 |
| actin1 promoter | 0.04 | 1.43 |
| polylinker | 1.43 | 1.46 |
| SmaI | 1.46 | 1.47 |
| Basta resistance | 1.47 | 2.03 |
| SmaI | 2.03 | 2.04 |
| polylinker | 2.03 | 2.08 |
| nos terminator | 2.08 | 2.34 |
| vector pSP72 | 2.34 | 4.74 |

TABLE 6
Positions of the characteristic elements of the
plasmid pRDP4 (unit: kilobase)

| | | |
|---|---|---|
| vector pSP72 | 0.0 | 0.04 |
| actin1 promoter | 0.04 | 1.43 |
| polylinker | 1.43 | 1.46 |
| tTA | 1.47 | 2.47 |
| polylinker | 2.47 | 2.52 |
| nos terminator | 2.52 | 2.78 |
| vector pSP72 | 2.78 | 5.18 |

TABLE 7
Positions of the characteristic elements of the
plasmid pTRE2 (unit: kilobase)

| | | |
|---|---|---|
| promoter with TRE | 0.00 | 0.44 |
| polylinker | 0.44 | 0.49 |
| SV40 terminator | 0.49 | 0.95 |
| Vector pUC | 0.95 | 3.10 |

TABLE 8
Positions of the characteristic elements of the
plasmid pRDP5 (unit: kilobase)

| | | |
|---|---|---|
| promoter with TRE | 0.00 | 0.44 |
| polylinker | 0.44 | 0.49 |
| Isum2A (ATG to terminator) | 0.49 | 2.73 |
| SV40 terminator | 2.73 | 3.19 |
| vector pUC | 3.19 | 5.34 |

### Example 3

### Construction of a Vector Comprising a Polynucleotide Encoding an ISUM2A Polypeptide Placed Under the Control of an Expression Control Sequence of the Inducible Activator Type

The expression system according to Example 3 constitutes an illustration of the expression system I summarized in Table 3, which is commented upon in the description.

The expression system according to Example 3 comprises two expression cassettes, respectively:
(i) an expression cassette comprising the isum2A gene placed under the control of a promoter containing the UAS sequence recognized by the GVG activator; and
(ii) an expression cassette encoding the GVG activator, placed under the control of the rice actin1 promoter, which is a strong and constitutive promoter in monocotyledons.
   A.1. Construction of the Vector pRDP2

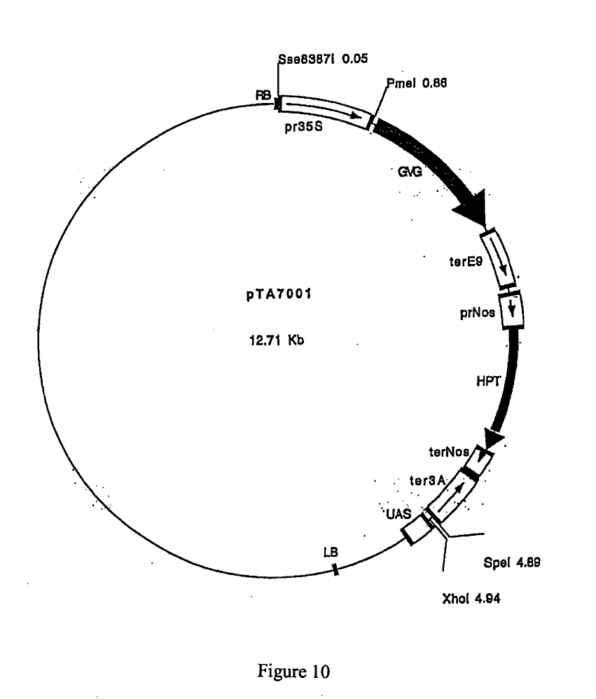

The starting vector is the plasmid pTA7001 described by AOYAMA et al. (1997) and which is represented in FIG. 10.

The vector pTA7001 is subjected to digestion with the Sse8387I and PmeI enzymes in order to excise the cauliflower mosaic virus 35S promoter.

After digestion, the sticky ends are filled in using Klenow polymerase.

The rice actin1 promoter is then amplified by PCR, from the plasmid pDM302 illustrated in FIG. 7.

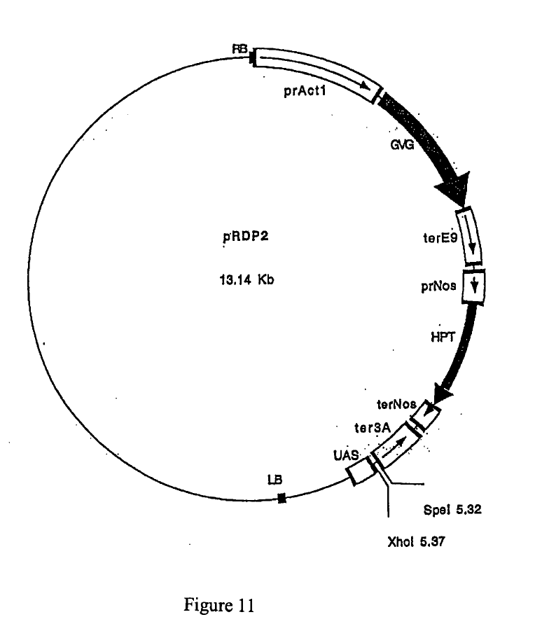

The rice actin1 promoter amplification product is then ligated into the predigested vector pTA7001, so as to construct the plasmid pRDP2 illustrated in FIG. 11.

A.2. Construction of the Vector pRDP3

The starting vector is the vector pRDP2 constructed according to the protocol above.

The vector pRDP2 is first subjected to digestion with the XhoI and SpeI restriction endonucleases.

After digestion, the sticky ends are filled in with Klenow polymerase.

The coding region of the isum2A gene is then amplified by PCR.

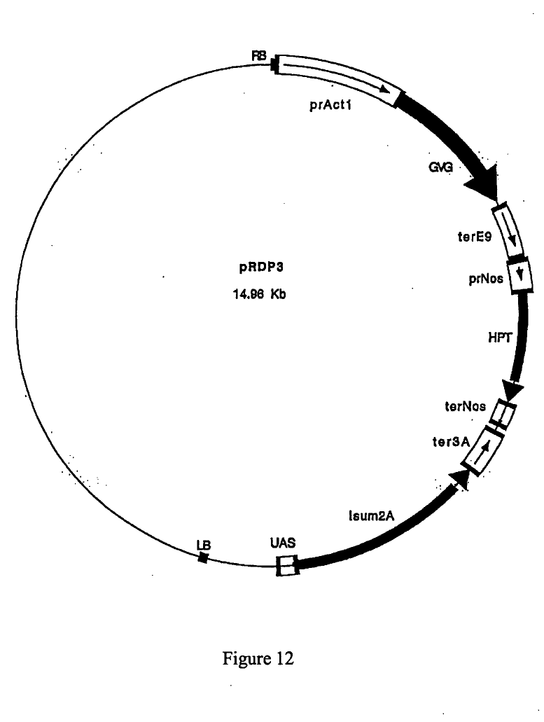

Finally, the isum2A gene amplification product is ligated into the open vector pRDP2 in order to construct the vector pRDP3 illustrated in FIG. 12.

The vector pRDP3 contains two expression cassettes, respectively:
(i) an expression cassette containing the isum2A gene placed under the control of the UAS sequence recognized by the GVG activator; and
(ii) an expression cassette comprising the sequence encoding the GVG activator placed under the control of the constitutive rice actin1 promoter.

The GVG gene encodes a protein from fusion between the DNA-binding domain of the GAL4 protein, the VP16 gene activator domain and the rat glucocorticoid receptor (GR).

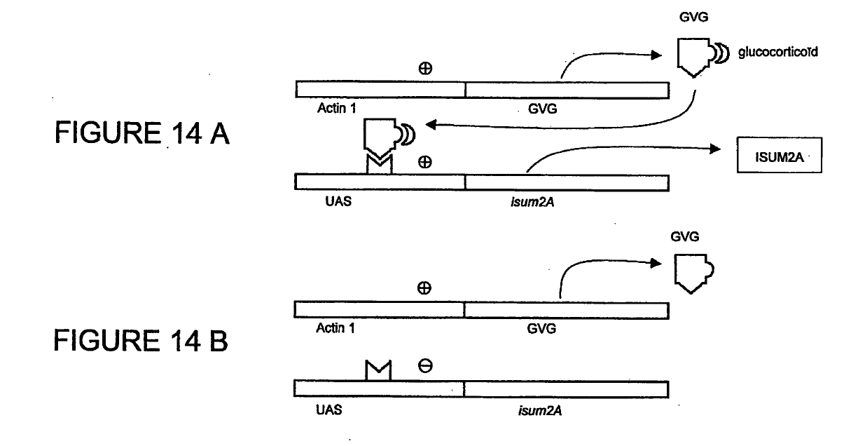

A scheme of the functioning of the inducible expression system described in the present example is represented in FIG. 14, even in the presence of glucocorticoid (FIG. 14A), or in the absence of glucocorticoid (FIG. 14B).

When the expression system according to Example 3 is used in the presence of the activator inducer signal consisting of a glucocorticoid hormone, the GVG hybrid transcription factor is transported into the nucleus and strongly activates all the promoters containing the UAS sequence (FIG. 14B).

Tables 9 to 11 below give a summary of the main characteristics of the various vectors used or prepared according to Example 3.

TABLE 9
Positions of the characteristic elements of the
plasmid pTA7001

| | | |
|---|---|---|
| Right border | 0.00 | 0.03 |
| Sse83871 | 0.05 | 0.05 |
| 35S promoter | 0.05 | 0.86 |
| Pmel | 0.87 | 0.87 |
| GVG | 0.87 | 2.18 |
| E9 terminator | 2.21 | 2.76 |
| Nos promoter | 2.78 | 3.11 |
| hygromycin resistance | 3.12 | 4.15 |
| Nos terminator | 4.15 | 4.40 |
| 3A terminator | 4.89 | 4.42 |
| Spel | 4.89 | 4.89 |
| Xhol | 4.94 | 4.94 |
| minimum TATA | 5.00 | 4.94 |
| 6 × GAL4 UAS | 5.20 | 5.00 |
| left border | 5.84 | 5.86 |
| pBI101 | 5.20 | 0.04 |

TABLE 10
Positions of the characteristic elements of the plasmid pRDP2

| | | |
|---|---|---|
| right border | 0.00 | 0.03 |
| actin1 promoter | 0.05 | 1.28 |
| GVG | 1.28 | 2.60 |
| E9 terminator | 2.62 | 3.18 |
| Nos promoter | 3.20 | 3.53 |
| hygromycin resistance | 3.54 | 4.56 |
| Nos terminator | 4.56 | 4.81 |
| 3A terminator | 5.31 | 4.84 |
| SpeI | 5.31 | 5.31 |
| XhoI | 5.36 | 5.36 |
| minumum TATA | 5.41 | 5.36 |
| 6 × GAL4 UAS | 5.61 | 5.41 |
| left border | 6.25 | 6.28 |
| pBI101 | 5.61 | 0.04 |

TABLE 11
Positions of the characteristic elements of the plasmid pRDP3

| | | |
|---|---|---|
| right border | 0.00 | 0.03 |
| actin1 promoter | 0.05 | 1.28 |
| GVG | 1.28 | 2.60 |
| E9 terminator | 2.62 | 3.18 |
| Nos promoter | 3.20 | 3.53 |
| hygromycin resistance | 3.54 | 4.56 |
| Nos terminator | 4.56 | 4.81 |
| 3A terminator | 5.31 | 4.84 |
| Isum2A | 7.18 | 5.31 |
| minimum TATA | 7.23 | 7.18 |
| 6 × GAL4 UAS | 7.44 | 7.23 |
| left border | 8.08 | 8.10 |
| pBI101 | 7.44 | 0.04 |

### Example 4

### Obtaining Plants Transformed with a Nucleic Acid Comprising a Polynucleotide Which Allows the Production of an ISUM2A Polypeptide

The transformation of a plant with the aim of obtaining a stable expression of the polynucleotide of interest in the transformed plant is necessary in order to ensure long-lasting modification of the maize grain. Experiments were carried out with a construct of vectors described in Examples 2 and 3.

A.1. Particle Gun

The method used is based on the use of a particle gun identical to that described by FINER (1992).

The target cells are rapidly dividing undifferentiated cells which have conserved an ability to regenerate whole plants. This type of cell makes up the maize embryogenic callus (referred to as type II callus). These calluses are obtained from immature embryos of the HiII genotype according to the method and on the media described by ARMSTRONG (1994) MAIZE HANDBOOK; 1994, M. FREELING, V. WALBOT EDS.; PP 665-671. Fragments of such calluses, having a surface area of from 10 to 20 mm², were placed, 4 h before bombardment, in a proportion of 16 fragments per dish, at the center of a Petri dish containing a culture medium identical to the initiating medium, supplemented with 0.2 M of mannitol+0.2 M of sorbitol. The plasmids described in the examples above and carrying the genes to be introduced are purified on a Qiagen^{R} column according to the manufacturer's instructions. They are then precipitated onto particles of tungsten (M10) according to the protocol described by KLEIN (1987). The particles thus coated are projected onto the target cells using the gun and according to the protocol described by J. FINER (1992). The dishes of calluses thus bombarded are then sealed with Scellofrais^{R} and then grown in the dark at 27° C. The first subculturing takes place 24 h later, and then every fifteen days for 3 months on medium identical to the initiating medium supplemented with a selective agent. After 3 months, or sometimes earlier, calluses are obtained, the growth of which is not inhibited by the selective agent, and which are usually and mainly made up of cells resulting from the division of a cell which has integrated into its genetic inheritance one or more copies of the selection gene. The frequency of production of such calluses is approximately 0.8 callus per bombarded dish.

These calluses are identified, individually separated, multiplied, and then cultivated so as to regenerate plantlets, by modifying the hormone and osmotic balance of the cells according to the method described by VAIN et al. (1989). These plants are then acclimatized in a greenhouse, where they can be crossed so as to obtain hybrids or selfpollinated.

A.2. Transformation with Agrobacterium

Another transformation technique which can be used in the context of the invention employs Agrobacterium tumefaciens, according to the protocol described ISHIDA et al. (1996), in particular using immature embryos of 10 days post-pollination. All the media used are referenced in the cited reference. The transformation begins with a coculturing phase in which the immature embryos of the maize plants are brought into contact for at least 5 min with Agrobacterium tumefaciens LBA 4404 containing the superbinary vectors. The superbinary plasmid is the result of homologous recombination between an intermediate vector carrying the T-DNA containing the gene of interest and/or the selection marker derived from the plasmids described in the above examples, and the vector pSB1 from Japan Tobacco (EP 672 752) which contains: the virB and virG genes of the plasmid pTiBo542 present in the supervirulent A281 strain of Agrobacterium tumefaciens (ATCC 37349) and a homologous region found in the intermediate vector which allows this homologous recombination. The embryos are then placed on LSA medium for 3 days in the dark at 25° C. A first selection is carried out on the transformed calluses: the embryogenic calluses are transferred onto LSD5 medium containing phosphinothricin at 5 mg/l and cefotaxim at 250 mg/l (elimination or limitation of the contamination with Agrobacterium tumefaciens). This step is carried out for 2 weeks in the dark and at 25° C. The second selection step is carried out by transferring the embryos which have developed on LSD5 medium onto LSD10 medium (phosphinothricin at 10 mg/l) in the presence of cefotaxim, for 3 weeks under the same conditions as previously. The third selection step consists in excising the type I calluses which remain white (fragments 1 to 2 mm) and in transferring them, for 3 weeks in the dark and at 25° C., onto LSD 10 medium in the presence of cefotaxim.

The regeneration of the plantlets is carried out by excising the type I calluses which have proliferated and transferring them onto LSZ medium in the presence of phosphinothricin at 5 mg/l and cefotaxim, for 2 weeks at 22° C. and under continuous light.

The plants which have regenerated are transferred onto RM+G2 medium containing 100 mg/l of Augmentin for 2 weeks at 22° C. and under continuous light, for the development step. The plants obtained are then transferred into a phytotron for the purpose of acclimatizing them.

### Example 5

### Biochemical Analyses

Biochemical analyses were carried out on maize grains obtained after self-pollination of heterozygous plants carrying the emb8516 mutation. Thus, the descendants are made up of grains of wild-type phenotype with an embryo (either wild-type homozygotes, heterozygotes), or of mutant phenotype without an embryo (mutant homozygotes).

These grains were sorted manually (visual sorting according to the phenotype) so as to separate those with a mutant phenotype from those with a wild-type phenotype.

The methods used on these grains to measure the various parameters (solids content, assaying of crude ash, EWERS assaying of starch, assaying of water-soluble fats, assaying of amino acids, etc.) may be based on the following approved ‘NFV’ or experimental ‘XPV’ AFNOR standards (available on the Internet site <http://www.affior.fr>, on-line standards):
Residual solids content (MSR 4H-130): NFV 03-708 March 1976. Maize: determination of the water content on whole grains and on ground grains. The method implemented in the present case does not use a refrigerated mill; for this reason, it is not true solids content, but simply a residual solids content on the grinding intended to be analyzed.
Assaying of crude ash (ash (2)): NFV 18-101 October 1977. Animal Feedstuffs Commission: assaying of crude ash.
EWERS assaying of starch (Ewers starch (2)): 3rd directive of the European Community with corrigendum ECOJ of Nov. 27, 1980. Assaying of starch, polarimetric method.
Assaying of fats with hydrolysis (MGH (2)): NFV 18-117 August 1997. Animal feedstuffs. Assaying of fats. Method B.
Assaying of walls (walls (2)): XP V 18-111 January 1998. Animal feedstuffs.
   Determination of the content of water-insoluble plant walls.
Assaying of amino acids (amino acids). The methods used are as follows:
   XIP V 18 113 January 1998. Animal feedstuffs. Assaying of amino acids.
   XP V 18 114 January 1998. Animal feedstuffs. Assaying of tryptophan.
Assaying of total nitrogenous materials: DUMAS method: NFV 18-120.

The results of the analyses carried out on the homozygous material comprising the Emb8516 mutation (Isum2 gene) and the corresponding wild-type control are represented in the table below:

TABLE 12
The data in the table are expressed in g/kg SC

| | | |
|---|---|---|
| Wild-type | | Differential |
| (control) | Emb8516 | (%)/control |

| | | | |
|---|---|---|---|
| Solids content (SC) in | 888.60 | 885.90 | −0.30 |
| g/kg of meal | | | |
| Wall content | 115.10 | 143.00 | 24.24 |
| Ewers starch | 678.40 | 719.50 | 6.06 |
| Hydrolyzable fats | 63.60 | 20.70 | −67.45 |
| Ash (mineral) | 16.30 | 11.60 | −28.83 |
| Ajinomoto SC in | 902.70 | 892.00 | −1.19 |
| g/kg of meal | | | |
| Ajinomoto total | 115.87 | 107.62 | −7.12 |
| nitrogenous material | | | |
| Lysine | 3.43 | 3.03 | −11.66 |
| Threonine | 4.21 | 3.81 | −9.50 |
| Methionine | 2.10 | 1.79 | −14.76 |
| Cysteine | 2.55 | 2.47 | −3.14 |
| Methionine + Cysteine | 4.54 | 4.26 | −6.17 |
| Tryptophan | 0.85 | 0.77 | −9.41 |
| Alanine | 8.64 | 7.74 | −10.42 |
| Arginine | 5.43 | 4.48 | −17.50 |
| Aspartic acid | 7.64 | 7.62 | −0.26 |
| Glutamic acid | 21.38 | 18.83 | −11.93 |
| Glycine | 4.43 | 3.92 | −11.51 |
| Histidine | 3.43 | 3.14 | −8.45 |
| Isoleucine | 4.10 | 3.70 | −9.76 |
| Leucine | 14.29 | 12.78 | −10.57 |
| Phenylalanine | 5.76 | 5.04 | −12.50 |
| Serine | 5.65 | 4.93 | −12.74 |
| Tyrosine | 3.77 | 3.48 | −7.69 |
| Valine | 5.65 | 5.27 | −6.73 |
| Total amino acids | 103.32 | 92.81 | −10.17 |

These analyses show that there is no difference in terms of the solids content of the grains.

On the other hand, the absence of embryo associated with the Emb8516 mutation results in a large decrease in the content of hydrolyzed fats (less than one third of the value of the control) and of mineral ash (decrease of 28.8%), and a slight decrease in total amino acids (10.2%). These data confirm the importance of the embryo as a source of fats and minerals and, indirectly, the effect of negative regulation of the expression of the ISUM2 polypeptide on the quality in terms of oil of the mature grain.

These decreases in fats, ash (minerals) and amino acids are, moreover, compensated for by an increase in the wall content (24.2%) and the starch content (6.1%), which confirms the close relationship which exists between development of the embryo and development of the albumen and the possibility of modulating the agronomic qualities of the embryo and/or the albumen as a function of the desired applications (semolina production or starch production).

These experiments therefore confirm the advantage of using the nucleic acid and polypeptide sequences according to the invention involved in the development of the embryo and/or the albumen, for modulating the agronomic qualities of the mature grain.

TABLE 13

| SEQUENCES | |
|---|---|
| SEQ ID NO. | Description |
| 1 | HD5 × HD7 genomic nucleic acid |
| 2 | A188 genomic nucleic acid |
| 3 | HD5 × HD7 cDNA |
| 4 | A188 cDNA |
| 5 | ISUM2A polypeptide from HD5 × HD7 |
| 6 | ISUM2A polypeptide from A188 |
| 7 | G2422 insertion sequence |
| 8 | Primer Mu16 |
| 9 | Primer adap MseI |
| 10 | Primer GW3 |
| 11 | Primer GW3b |
| 12 | Primer GW4 |
| 13 | Primer GW4b |
| 14 | Primer Mu15 |
| 15 | Primer AP1 |
| 16 | Primer AP2 |
| 17 | Primer Isum2a |
| 18 | Primer Isum2b |
| 19 | Primer Isum2c |
| 20 | Primer Isum2e |
| 21 | Primer Isum2k |
| 22 | Primer Isum2l |
| 23 | Primer 8516g |
| 24 | Primer OMuA |

### Documents Cited

All sequences, patents, patent applications or other published documents cited anywhere in this specification are herein incorporated in their entirety by reference to the same extent as if each individual sequence, publication, patent, patent application or other published document was specifically and individually indicated to be incorporated by reference.

An et al. (1986), Plant Physiol., 81: 86-91.

AOYAMA, T., AND CHUA, N-H. (1997) A glucocorticoid-mediated transcriptional induction system in transgenic plants. Plant J. 11:605-612

AOYAMA T et al., (1997) The Plant Journal, vol.11 (3):605-612.

AUSUBEL F M, BRENT R, KINGSTON R E, MOORE D D, SEIDMAN J G, SMITH J A, STRUHL K Editors, Current protocols in Molecular Biology, Wiley Interscience. (1994).

AUSUBEL T et al., 1989. Current Protocols in Molecular Biology, Green Publishing Associates and Wiley Interscience, N.Y.

BARLOY D, DENIS L, BECKERT M (1989) Comparison of the aptitude for anther culture in some androgenetic doubled haploid maize lines. Maydica 34: 303-308

BERBAL, 1984.

BROWN et al. (1979), Methods Enzymol., 68:109-151.

BEAUCAGE et al. (1981), Tetrahedron Lett., 22:1859-1862.

BECHTOLD et al. 1993, Comptes-Rendus de l'Académie des Sciences Série III Sciences de la Vie. 316:10, 1194-1199.

BEVAN et al., Nucleic Acids Research, vol.12:8711-8721.

BOUCHEZ D., Comptes Rendus de l'Académie des Sciences Série III Sciences de la Vie, 1993 316:10, 1188-1193

CARRINGTON ET FREED. J. VIROL. (1990), 64(4): 1590-1597

CHUPEAU et al. Biotechnology (1989), 7(5): 503-508

CAO et al. (1992), Plant cell reports, vol. 11(11): 586-591.

DEPICKER et al., 1992 [PLEASE COMPLETE]

DEVIC M, ALBERT S, DELSENY M, ROSCOE T J (1997) Efficient PCR walking on plant genomic DNA. Plant Physiol Biochem 35: 331-339

ELROY-STEIN et al. PNAS USA (1989), 86: 6126-6130.

FRANCK ET AL. (1980), Cell, 21:285-294

FREY M, STETTNER C, GIERL A (1998). A general method for gene isolation in tagging approaches: amplification of insertion mutagenised sites (AIMS). Plant J 13: 717-721

FROMM M. et al. (1990), Biotechnology, 8:833-839

FINER J. (1992) Plant Cell Report, 11:323-328.

GORLACH J, VOLRATH S, KNAUF-BEITER G, HENGY G, BECKHOVE U, KOGEL K H, OOSTENDORP M, STAUB T, WARD E, KESSMANN H, RYALS J. (1996) Benzothiadiazole, a novel class of inducers of systemic acquired resistance, activates gene expression and disease resistance in wheat. Plant Cell 8:629-43

GAIT (ed.), (1984). Nucleic Acid Hybridization.

GLOVER (ed.), 1985. DNA Cloning: A Practical Approach, Volumes I and II Oligonucleotide Synthesis, MRL Press, Ltd., Oxford, U.K.

GALLIE et al. Molecular Biology of RNA (1989), 237-256.

GERDES J T, Tracy W F (1993). Pedigree diversity within the Lancaster surecrop heterotic group of maize. Crop Sci 33: 334-337

GUERCHE ET AL. (1987), Mol. Gen. Genet., 206:382

HANAHAN D (1983). Studies on transformation of Escherichia coli with plasmids. J Mol Biol 166: 557-580.

HECKEL T, WERNER K, SHERIDAN W F, DUMAS C, ROGOWSKY P M (1999) Novel phenotypes and developmental arrest in early embryo specific (emb) mutations of maize. Planta, 210: 1-8.

HAMES and HIGGINS, 1985. Nucleic Acid Hybridization: a practical approach, Hames & Higgins Ed. IRL Press, Oxford.

HAJDUKIEWICZ, P. SVAB. Z. AND MALIGA P. Plant Mol. Biol. 25 (6), 989-994 (1994).

HORSCH R. B. Commercialization of genetically engineered crops. The production and uses of genetically transformed plants. Chapman 1 Hall LTD. London UK 19947, 99-103.

HECKEL T et al., 1999, Planta, vol. 210: 1-8

HOUBEN WEIL, (1974).In Methode der Organischen Chemie, E.Wunsh ed., volume 15-I and 15-II, Thieme, Stuttgart.

ISHIDA ET AL. (1996), Nature Biotechnology, vol. 14: 745-750.

JEFFERSON, 1987, Plant Molecular Biology Reporter, vol.5:387-405.

JOBLING et al. Nature (1987), 325 : 622-625.

KADDICK M X et al., (1998), Nature Biotechnology, vol.16:177-180.

KOZBOR et al., (1983), Hybridoma, vol.2 (1):7-16.

KOHLER G and MILSTEIN C;, (1975), Nature, volume 256:495

KOOTER, J M., MATZKE, M A., AND MEYER, P. (1999) Listening to the silent genes: transgene silencing, gene regulation and pathogen control. Trends Plant Sci. 4, 430-437

LEGER O J et al., (1997), Hum Antibodies, vol.8 (1):3-16

McCARTY, D R., CARSON, C B., STINARD, P S., AND ROBERTSON, D S. (1989) Molecular analysis of viviparous-1: an abscisic acid-insensitive mutant of maize. Plant Cell, 1:523-532

MACEJACK et al. Nature (1991), 353 : 90-94

Molina A, Hunt M D, Ryals J A (1998) Impaired fungicide activity in plants blocked in disease resistance signal transduction. Plant Cell 10:1903-14

Martinez, A., Sparks, C., Hart, C A., Tompson, J., and Jepson, I. (1999) Ecdysone agonist inducible transcription in transgenic tobacco plants. Plant J. 19:97-106

McNELLIS T W, 1998, The Plant Journal, vol.14 (2): 247-257

MERRIFIELD R B, (1965a), Nature, vol.207 (996):522-523

MERRIFIELD R B, (1965b), Science, vol.150 (693):178-185

MARTINEAU P et al., (1998), J. Mol. Biol. vol.280(l):117-127.

NARANG et al. (1979), Methods Enzymol., 68: 90-98.

NEUHAUS ET AL. Theoretical and Applied Genet. (1987), 75(1): 30-36

RIDDER R. et al., (1995), Biotechnology (NY), vol.13 (3):255-260.

REINMANN K A et al. (1997), Aids Res. Hum retroviruses, vol.13 (11):933-943.

SAMBROOK, J. FRITSCH, E. F., and T. Maniatis, 1989. Molecular cloning: a laboratory manual. 2ed. Cold Spring Harbor Laboratory, Cold spring Harbor, N.Y.

SANCHEZ PESCADOR, (1988), J. Clin. Microbiol., 26 (10):1934-1938.

SALTER M G et al., 1998, vol.16 (1): 127-132

SCHOCHER ET AL. Biotechnology (1986), 4: 1093-1096

URDEA et al. (1988), Nucleic Acids Research, 11:4937-4957.

URDEA et al. (1991), Nucleic Acids Research, 24: 197-200.

VAIN P et al., (1989), Plant Cell Tissue and Organ Culture, 18: 143-151.

WATSON et al. (1994)

WASSENEGGER, M., AND PÉLISSIER, T. (1998) A model for RNA-mediated gene silencing in higher plants Plant Mol. Biol. 37, 349-362

YANG F. MOSS L G, PHILIPS G N JR. Nat. Biotechnol. Oct. 14, 1996(10):1246-51.

YANG T T, CHENG L. KAIN S R, Nucleic acids Res., Nov. 15, 1996; 24(22):4592-3.

1. An isolated and purified nucleic acid comprising a nucleotide sequence selected from the group consisting of a nucleotide sequence encoding an ISUM2A polypeptide having an amino acid sequence that is at least 95% identical to SEQ ID NO:5, a nucleotide sequence encoding an ISUM2A fragment having an amino acid sequence identical to at least 100 consecutive amino acids of SEQ ID NO:5, a nucleotide sequence encoding an ISUM2A polypeptide having an amino acid sequence that is at least 95% identical to SEQ ID NO:6, and a nucleotide sequence encoding an ISUM2A fragment having an amino acid sequence identical to at least 100 consecutive amino acids of SEQ ID NO:6, and complements thereof.

2. The nucleic acid of claim 1, comprising a nucleotide sequence selected from the group consisting of a nucleotide sequence having at least 95% nucleotide identity with the nucleotide sequence SEQ ID NO:1, a nucleotide sequence complementary to a nucleotide sequence having at least 95% nucleotide identity with the nucleotide sequence SEQ ID NO:1, a nucleotide sequence identical to at least 300 consecutive nucleotides of SEQ ID NO:1, and a nucleotide sequence complementary to a nucleotide sequence identical to at least 300 consecutive nucleotides of SEQ ID NO:1.

3. The nucleic acid of claim 1, comprising a nucleotide sequence selected from the group consisting of a nucleotide sequence having at least 95% nucleotide identity with the nucleotide sequence SEQ ID NO:2, a nucleotide sequence complementary to a nucleotide sequence having at least 95% nucleotide identity with the nucleotide sequence SEQ ID NO:2, a nucleotide sequence identical to at least 300 consecutive nucleotides of SEQ ID NO:2, and a nucleotide sequence complementary to a nucleotide sequence identical to at least 300 consecutive nucleotides of SEQ ID NO:2.

4. The nucleic acid of claim 1, comprising a nucleotide sequence selected from the group consisting of a nucleotide sequence having at least 99% nucleotide identity with the nucleotide sequence SEQ ID NO:3, a nucleotide sequence complementary to a nucleotide sequence having at least 99% nucleotide identity with the nucleotide sequence SEQ ID NO:3, a nucleotide sequence identical to at least 300 consecutive nucleotides of SEQ ID NO:3, and a nucleotide sequence, complementary to a nucleotide sequence identical to at least 300 consecutive nucleotides of SEQ ID NO:3.

5. The nucleic acid of claim 1, comprising a nucleotide sequence selected from the group consisting of a nucleotide sequence having at least 99% nucleotide identity with the nucleotide sequence SEQ ID NO:4, a nucleotide sequence complementary to a nucleotide sequence having at least 99% nucleotide identity with the nucleotide sequence SEQ ID NO:4, a nucleotide sequence identical to at least 300 consecutive nucleotides of SEQ ID NO:4, and a nucleotide sequence complementary to a nucleotide sequence identical to at least 300 consecutive nucleotides of SEQ ID NO:4.

6. The nucleic acid of claim 1 further comprising an expression control sequence operably linked to the nucleotide sequence.

7. The nucleic acid of claim 6, wherein the expression control sequence allows overexpression of the nucleotide sequence.

8. The nucleic acid of claim 6, wherein the expression control sequence is sensitive to the action of an inducer signal.

9. The nucleic acid of claim 6, wherein the expression control sequence is an inducible transcription- or translation-represssing sequence.

10. The nucleic acid of claim 6, wherein the expression control sequence is an inducible transcription- or translation-activating sequence.

11. The nucleic acid of claim 10, wherein the inducible activator polynucleotide is a polynucleotide encoding the GVG activator, operably linked to the rice actin 1 gene promoter.

12. An isolated and purified nucleic acid comprising at least 12 nucleotides that hybridizes specifically with a nucleic acid comprising a nucleotide sequence selected from the group consisting of a nucleotide sequence encoding an ISUM2A polypeptide having an amino acid sequence that is at least 95% identical to SEQ ID NO:5, a nucleotide sequence encoding an ISUM2A fragment having an amino acid sequence identical to at least 100 consecutive amino acids of SEQ ID NO:5, a nucleotide sequence encoding an ISUM2A polypeptide having an amino acid sequence that is at least 95% identical to SEQ ID NO:6, and a nucleotide sequence encoding an ISUM2A fragment having an amino acid sequence identical to at least 100 consecutive amino acids of SEQ ID NO:6, and complements thereof.

13. An isolated and purified nucleic acid that hybridizes specifically with a nucleic acid encoding an ISUM2A polypeptide, the sequence of which is selected from the group consisting of SEQ ID NOS:10, 11, 12, 13, 17, 18, 19, 20, 21, 22, and 23.

14. A method of detecting the presence of an ISUM2A polypeptide in a sample, comprising:
contacting
a sample prospectively comprising a nucleic acid encoding an ISUM2A polypeptide with
an isolated and purified probe nucleic acid comprising at least 12 nucleotides that hybridizes specifically with a nucleic acid comprising a nucleotide sequence selected from the group consisting of a nucleotide sequence encoding an ISUM2A polypeptide having an amino acid sequence that is at least 95% identical to SEQ ID NO:5, a nucleotide sequence encoding an ISUM2A fragment having an amino acid sequence identical to at least 100 consecutive amino acids of SEQ ID NO:5, a nucleotide sequence encoding an ISUM2A polypeptide having an amino acid sequence that is at least 95% identical to SEQ ID NO:6, and a nucleotide sequence encoding an ISUM2A fragment having an amino acid sequence identical to at least 100 consecutive amino acids of SEQ ID NO:6, and complements thereof,
under conditions that permit specific hybridization of probe and target nucleic acids.

15. A recombinant vector comprising the nucleic acid of claim 1.

16. A host cell transformed with the nucleic acid of claim 1 or with the recombinant vector of claim 15.

17. The host cell of claim 16, wherein the host cell is a plant cell.

18. The use of the nucleic acid of claim 1 for producing a transformed plant capable of producing grains with altered germ development.

19. The use of the nucleic acid of claim 6 for obtaining a transformed plant capable of producing grains rich in oil.

20. A plant transformed with the nucleic acid of claim 1 or with the recombinant vector of claim 15.

21. A transformed plant comprising a plurality of the host cells of claim 17.

22. The transformed plant of claim 21, wherein the transformed plant has been derived from a plant in which the two endogenous copies of the Isum2A gene each comprise at least one mutation which causes a deficiency in the production of an ISUM2A polypeptide of sequence SEQ ID NO:5 or 6.

23. A method for obtaining a transformed plant capable of producing grains with altered germ development, comprising:
a) transforming at least one plant cell with the nucleic acid of claim 6 or with a recombinant vector comprising the nucleic acid of claim 6;
b) selecting the transformed cells obtained in step (a) which have integrated into their genome at least one copy of the nucleic acid of claim 6;
c) regenerating a transformed plant from the transformed cells obtained in step (b).

24. The method of claim 23, in which at least one plant cell is transformed in step (a) with Agrobacterium tumefaciens containing the nucleic acid of claim 6 or with a recombinant vector comprising the nucleic acid of claim 6.

25. A transformed plant obtained by the method of claim 23.

26. A hybrid transgenic plant obtained by crossing the plant of claim 25.

27. A part of the transformed plant of claim 25.

28. A method for obtaining plant grains with altered germ development, comprising:
a) cultivating, until pollination,
a plant in which the two endogenous copies of the Isum2A gene each comprise at least one mutation which causes a deficiency in the production of an ISUM2A polypeptide, and into the genome of which has been introduced the nucleic acid of claim 9,
in the absence of the repressor inducer signal to which the repressor expression control sequence is sensitive;
b) bringing the transformed plant defined in (a) into contact with the repressor inducer signal to which the repressor expression control sequence is sensitive, for a period of time ranging from the beginning of pollination to the end of grain formation;
c) recovering the mature grains altered in germ development.

29. A method for obtaining plant grains with altered germ development, comprising:
a) cultivating, until pollination,
a plant in which the two endogenous copies of the Isum2A gene each comprise at least one mutation which causes a deficiency in the production of an ISUM2A polypeptide, and into the genome of which has been introduced the nucleic acid of claim 10,
in the presence of the activator inducer signal to which the activator expression control sequence is sensitive;
b) continuing the cultivation of the transformed plant defined in (a) in the absence of the activator inducer signal to which the activator expression control sequence is sensitive, from the period following pollination;
c) recovering the mature grains altered in germ development.

30. A seed altered in germ development obtained by the method of either claim 28 or 29.

31. A seed altered in germ development comprising the nucleic acid of claim 6.

32. A product of the seed of claim 30.

33. The product of claim 32, wherein the product is a starch.

34. The product of claim 32, wherein the product is a seed meal or an oil.

35. An isolated and purified ISUM2A polypeptide or an isolated and purified fragment an ISUM2A polypeptide encoded by a nucleic acid comprising a nucleotide sequence selected from the group consisting of a nucleotide sequence encoding an ISUM2A polypeptide having an amino acid sequence that is at least 95% identical to SEQ ID NO:5, a nucleotide sequence encoding an ISUM2A fragment having an amino acid sequence identical to at least 100 consecutive amino acids of SEQ ID NO:5, a nucleotide sequence encoding an ISUM2A polypeptide having an amino acid sequence that is at least 95% identical to SEQ ID NO:6, and a nucleotide sequence encoding an ISUM2A fragment having an amino acid sequence identical to at least 100 consecutive amino acids of SEQ ID NO:6, and complements thereof.

36. The polypeptide of claim 35 having an amino acid sequence that is at least 95% amino acid identical to SEQ ID NO:5 or SEQ ID NO:6.

37. An antibody directed against the polypeptide of claim 35.

38. A method for detecting the presence of the polypeptide of claim 35, in a sample, comprising:
(a) contacting the sample with an antibody directed against the polypeptide of claim 35; and
(b) detecting formation of an antigen/antibody complex.

39. A kit for detecting the polypeptide of claim 35, in a sample, comprising:
(a) an antibody directed against the polypeptide of claim 35; and
(b) optionally, one or more reagents required for the detection of the antigen/antibody complex.

40. The use of a nucleic acid or of an allelic variant of the nucleic acid of claim 1, in selection programs for obtaining plants with modified embyro size and/or development influencing the content of starch and/or of oil.

41. A method for selecting plants with modified embryo size and/or development, comprising:
(a) genotyping individual plants of a group of two or more plants using the nucleotide probe of claim 12; and
(b) selecting, from these plants (individuals), those which comprise a high frequency of favorable alleles associated with the size and/or development of the embryo.
